(19) United States
(12) Patent Application Publication
(10) Pub. No.: US 2021/0030719 A1
(43) Pub. Date: Feb. 4, 2021

(54) GRP94 INHIBITORS TO TREAT STEROID-INDUCED OCULAR HYPERTENSIONS AND GLAUCOMAS

(71) Applicants: UNIVERSITY OF SOUTH FLORIDA, Tampa, FL (US); THE UNIVERSITY OF KANSAS, Lawrence, KS (US)

(72) Inventors: Chad A. DICKEY (Deceased), (US); John KOREN, Tampa, FL (US); Laura J. BLAIR, Brandon, FL (US); Brian S.J. BLAGG, Niles, MI (US); Ricardo A. CORDOVA, Tampa, FL (US); Zheying SUN, Lutz, FL (US)

(21) Appl. No.: 16/971,994

(22) PCT Filed: Feb. 22, 2019
(86) PCT No.: PCT/US19/19196

§ 371 Date: Aug. 21, 2020

Related U.S. Application Data
(60) Provisional application No. 62/633,659, filed on Feb. 22, 2018.

(51) Int. Cl. A61K 31/4174; A61P 27/06; A61K 9/00; A61K 9/08; A61K 31/4439; A61K 31/506

(52) U.S. Cl. CPC A61K 31/4174; A61P 27/06; A61K 9/0048; A61K 31/506; A61K 9/08; A61K 31/4439; A61K 9/0019

(57) ABSTRACT

A method and composition for preventing, reducing, or treating steroid-induced ocular hypertension and steroid-induced glaucoma using a selective Grp94 inhibitor is presented. The Grp94-selective inhibitor can include methyl 2-(2-(1(4-bromobenzyl)-1H-imidazol-2-yl)ethyl)-3-chloro-4,6-dihydroxybenzoate (4-Br—BnIm) or a derivative thereof. The Grp94-selective inhibitor can be administered prior to, during, or after administration of a steroid to the patient.

### STATEMENT OF GOVERNMENTAL INTEREST

This invention was made with government support under Grant No. EY024232 awarded by the National Institutes of Health. The Government has certain rights in the invention.

### FIELD

This disclosure relates to treatment of steroid-induced ocular hypertensions and glaucoma. Specifically, the disclosure provides a method and compositions for treating steroid-induced ocular hypertensions and glaucoma by selectively inhibiting Grp94.

### BACKGROUND

Glaucoma is a collection of diseases which result in damage to the optic nerve. Despite advances in treatment strategies, glaucoma remains the second leading cause of blindness worldwide, affecting 70 million people worldwide (Quigley, H. A. et al. Br J Ophthalmol, 2006. 90(3): p. 262-7). Damage to the optic nerve and surrounding retinal cells is often the result of increased intraocular pressure (IOP). Elevated IOP develops from an imbalance of aqueous humor production and clearance. Current treatments include topical and surgical options to reduce IOP, but there are currently no therapeutic options to treat the underlying cause of the disease, and a large percentage of patients on IOP-lowering drugs eventually reach blindness. The lack of therapeutic options for glaucoma is, in part, due to the diverse nature of how glaucoma develops as well as a lack of understanding into the origin of these diseases.

For the majority of glaucoma cases, the cause of IOP elevation is unknown, but the inherited early-onset subtype of open angle glaucoma (3-5% of all OAG cases, ˜3 million patients) is caused by a toxic-gain-of-function resulting from mutations of the protein myocilin, particularly non-synonymous mutations particularly within the olfactomedin (OLF) domain (Gong, G., et al., Hum. Mol. Genet., 2004. 13: p. R91-102). Coding mutations lead to mutant protein sequestered in the ER in an aggregated form, causing TM cell cytotoxicity. Overexpression of wild type (WT) myocilin does not induce glaucoma in mouse models (Gould, D. B., et al., Mol Cell Biol, 2004. 24(20): p. 9019-25; and Zillig, M., et al., Invest Ophthalmol Vis Sci, 2005. 46(1): p. 223-34), suggesting the levels of WT myocilin alone are not sufficient to induce glaucoma. In addition, silencing or ablating myocilin is well tolerated in animal models (Kim, B. S., et al., Mol. Cell Biol., 2001. 21(22): p. 7707-13), myocilin knockout (Myoc-KO) mice are viable, and individuals harboring far N-terminal truncation mutations do not develop glaucoma (Lam, D. S., et al., Invest. Ophthalmol. Vis. Sci., 2000. 41(6): p. 1386-91), suggesting that the elimination of mutant myocilin is a potential strategy to treat glaucoma.

Prior to the connection to heritable glaucoma, levels of myocilin were found to be dramatically increased following the application of glucocorticoids. This finding suggested that myocilin was linked to secondary forms of glaucoma. Chronic, topical, and ocular regimens of glucocorticoids, such as dexamethasone (dex), can induce these secondary forms of glaucoma. If a patient is susceptible to this form of glaucoma, cessation of the steroid regimen can reduce associated phenotypes, such as elevated IOP. However, some patients afflicted with steroid-induced glaucoma present irreversible phenotypes that result in blindness.

Recently, it was demonstrated that, despite the responsiveness of myocilin to dex, steroid regimens can induce an elevated IOP phenotype in mice lacking myocilin (myoc -/-, myoc-KO), which demonstrated that myocilin was not necessary for the development of steroid-induced glaucoma (Lam, D. S., et al., Invest. Ophthalmol. Vis. Sci., 41(6), 1386-91 (2000)). Interestingly, analysis of the myoc-KO mice and wild type WT mice treated with dex demonstrated elevations in ER stress genes as well as altered expression of ECM components. These results are particularly intriguing because mutant myocilin produced similar elevations in ER stress markers, IOP, and alterations to ECM proteins. Thus, dex impacts glaucoma development in a manner highly similar to that of mutant myocilin; mechanisms potentially connected through the activation of ER stress and altered ECM.

Over 2.7 million Americans suffer from primary open angle glaucoma (POAG), the most common glaucoma subtype leading to blindness. The most prevalent and clinically addressable risk factor is elevated intraocular pressure (IOP) which precedes vision loss. Dysregulation of aqueous humor outflow due to trabecular meshwork (TM) extracellular matrix dysfunction is thought to underlie ocular hypertension. The resulting IOP elevation leads to irreversible optic nerve and retinal ganglion cell (RGC) death and blindness.

Molecular chaperones play a critical role in cellular homeostasis by modulating the folding, stabilization, activation, and degradation of protein substrates (Hartl, F. U. Nature 381, 571-580, (1996); Hartl, F. U., et al. Nature 475, 324-332, (2011)). Heat shock proteins (Hsps) represent a class of molecular chaperones that are overexpressed in response to cellular stress, including elevated temperatures (Whitesell, L., et al. Curr. Cancer Drug Tar. 3, 349-358, (2003); Whitesell, L. et al. Nat. Rev. Cancer 5, 761-772, (2005)). Amongst the various Hsps, the 90 kDa heat shock proteins (Hsp90) are considered promising anti-cancer targets due to the role they play in the maturation of various signaling proteins (Bishop, S. C., et al. Curr. Cancer Drug Tar. 7, 369-388, (2007); Blagg, B. S. J. et al. Med. Res. Rev. 26, 310-338, (2006); Chiosis, G., et al. Drug Discov. Today 9, 881-888, (2004)). Hsp90 is both overexpressed and activated in transformed cells, which allows for the attainment of high differential selectivities for Hsp90 inhibitors (Whitesell, L., et al. Curr. Cancer Drug Tar. 3, 349-358, (2003); Whitesell, L. et al. Nat. Rev. Cancer 5, 761-772, (2005); Zhang, H. et al. J. Mol. Med. 82, 488-499, (2004)). In addition, Hsp90-dependent substrates are directly associated with all six hallmarks of cancer, and thus, through Hsp90 inhibition, multiple oncogenic pathways are simultaneously disrupted, resulting in a combinatorial attack on cancer (Zhang, H. et al. J. Mol. Med. 82, 488-499, (2004); Hanahan, D. et al. Cell 100, 57-70, (2000) Hanahan, D. et al. Cell 144, 646-674, (2011); Workman, P. Cancer Lett. 206, 149-157, (2004); Workman, P., et al. Ann. NY Acad. Sci. 1113, 202-216, (2007)).

Hsp90 contains an atypical nucleotide binding pocket, which allows for the development of selective inhibitors. (Dutta, R. et al. Trends Biochem. Sci. 25, 24-28, (2000)). Several of these Hsp90 N-terminal inhibitors have progressed into clinical trials, however cardiovascular, ocular, and/or hepatotoxicities have been observed. (Biamonte, M. A. et al. J. Med. Chem. 53, 3-17, (2010); Holzbeierlein, J., et al. Curr. Oncol. Rep. 12, 95-101, (2010); Kim, Y. S. et al. Curr. Top. Med. Chem. 9, 1479-1492, (2009)).

Pan-Hsp90 inhibition is likely the cause for these effects, as clinical inhibitors target all four human isoforms; Hsp90α, Hsp90β, Trap1 and Grp94. Hsp90α (inducible) and Hsp90β (constitutively active) are the cytosolic isoforms, whereas tumor necrosis factor receptor associated protein (TRAP1) is localized to the mitochondria, and glucose-regulated protein, Grp94, resides in the endoplasmic reticulum (Sreedhar, A. S., et al. FEBS Lett.562, 11-15, (2004)). Little is known about the client protein selectivity manifested by each of the four isoforms, and this gap in understanding may underlie the toxicity concerns that have arisen in clinical trials. Despite the clinical significance of Hsp90 inhibition, little investigation towards the development of isoform-selective inhibitors has been pursued to delineate isoform-dependent substrates, or as an opportunity to reduce the side effects that result from pan-inhibition.

Unlike the cytosolic chaperones, Hsp90α and Hsp90β, which have been well-studied, little is known about TRAP1 and Grp94. At present, no isoform specific clients have been described for TRAP-1, In fact, neither the crystal nor the solution structure has been solved. In contrast, Grp94 co-crystal structures have recently been determined, and demonstrate this isoform to exhibit a unique secondary binding pocket that may provide an opportunity to develop isoform-selective inhibitors (Dollins, D. E., J. Biol. Chem.280, 30438-30447, (2005); Dollins, D. E., et al. Mol. Cell 28, 41-56, (2007); Immormino, R. M. et al. J. Biol. Chem. 279, 46162-46171, (2004); Immormino, R. M. et al. J. Mol. Biol. 388, 1033-1042, (2009); Krukenberg, K. A., et al. Protein Sci. 18, 1815-1827, (2009); Krukenberg, K. A. et al. J. Mol. Biol. 390, 278-291, (2009); Soldano, K. L., et al. J. Biol. Chem. 278, 48330-48338, (2003)). Unlike TRAP-1, several substrates dependent upon Grp94 have been identified and include Toll-like receptors (TLR1, TLR2, TLR4 and TLR9), integrins (CD11a, CD18, CD49d, α4, β7, αL and β2), IGF-I and -II and immunoglobulins (Marzec, M., et al. BBA-Mol. Cell Res. 1823, 774-787, (2012); Maynard, J. C. et al. Dev. Biol. 339, 295-306, (2010); McLaughlin, M. et al. Brit. J. Pharmacol. 162, 328-345, (2011); Wanderling, S. et al. Mol. Biol. Cell 18, 3764-3775, (2007); McLaughlin, M., et al. Neuroimmunol. 203, 268, (2008); Olson, D. L., et al. Molecular Cancer Therapeutics 4, 91-99, (2005); Ostrovsky, O., et al. Biochimica et Biophysica Acta (BBA)-Molecular Cell Research 1803, 333-341, (2010); Randow, F. et al. Nat Cell Biol 3, 891-896, (2001); Saitoh, T. et al. Molecular Pharmacology 62, 847-855, (2002); Yang, Y. et al. Immunity 26, 215-226, (2007)).

Since these clients play key roles in cell-to-cell communication and adhesion, Grp94-selective inhibitors may disrupt malignant progression by preventing metastasis, migration, immunoevasion and/or cell adhesion (Ostrovsky, O., et al. Biochimica et Biophysica Acta (BBA)-Molecular Cell Research 1803, 333-341, (2010); Randow, F. et al. Nat Cell Biol 3, 891-896, (2001); Saitoh, T. et al. Molecular Pharmacology 62, 847-855, (2002); Yang, Y. et al. Immunity 26, 215-226, (2007); Belfiore, A., et al. Biochimie 81, 403-407, (1999); Chavany, C. et al. Journal of Biological Chemistry 271, 4974-4977, (1996); Moorehead, R. A., et al. Oncogene 22, 853-857, (2003); Supino-Rosin, L., et al. Journal of Biological Chemistry 275, 21850-21855, (2000)). Interestingly, many of these Grp94-dependent clients have also been identified as key contributors to inflammatory disorders such as rheumatoid arthritis, diabetes, and asthma (McLaughlin, M., et al. Neuroimmunol. 203, 268, (2008); Zuany-Amorim, C., et al. Nat Rev Drug Discov 1, 797-807, (2002); McLaughlin, M. et al. British Journal of Pharmacology 162, 328-345, (2011); Randow, F. et al. Nat Cell Biol 3, 891-896, (2001)). Therefore, the ability to develop a Grp94-selective inhibitor may not only provide a new paradigm for Hsp90 inhibition but may also provide new opportunities for the treatment of diseases other than cancer.

The biological roles manifested by Grp94 have been primarily elucidated through the use of RNAi induced Grp94 knockdown, immunoprecipitation experiments, or through pan-inhibition of all four Hsp90 isoforms. A selective small molecule inhibitor of Grp94 would provide an alternative and powerful method for further elucidation of the roles manifested by Grp94, as well as the identity of other Grp94-dependent processes/substrates. Recently, the co-crystal structures of the chimeric inhibitor, radamide (RDA), bound to the N-terminal domain of both the yeast ortholog of cytosolic Hsp90 (yHsp82N, PDB: 2FXS) and the canine ortholog of Grp94 (cGrp94NΔ41, PDB: 2GFD) were described (Immormino, R. M. et al. J. Mol. Biol. 388, 1033-1042, (2009)). Utilizing a structure-based approach that relied upon these co-crystal structures, a new class of inhibitors that target Grp94 has been developed.

Small-molecule inhibition of Hsp90, including Grp94, is in clinical development for a number of diseases (Dickey C A, et al. J Clin Invest 117(3), 648-658, (2007); Kamal A, et al. Trends Mol Med 10(6), 283-290, (2004); Neckers L et al. Clin Cancer Res 18(1), 64-76, (2012)).

Analogous inhibition of the paralog Hsp90 is under investigation as therapeutic strategy for many diseases including cancers and Alzheimer's disease (Dickey CA, et al. (2007); Neckers L et al. Clin Cancer Res 18(1), 64-76; Luo W, et al. Proc Nall Acad Sci U S A 104(22), 9511-9516, (2007)). Importantly, depletion of Grp94, while lethal during development, has no obvious consequence in adults (Maynard J C, et al. Dev Biol 339(2), 295-306, (2010)). Grp94 is structurally similar to cytosolic Hsp90, but lacks known co-chaperones and has very few known clients; the limited list includes immunoglobulins, integrins and toll-like receptors (Marzec M, et al. Biochim Biophys Acta 1823(3), 774-787, (2012); Melnick J, et al. Nature 370(6488), 373-375, (1994); Liu Y, et al. J Cell Biol 182(1), 185-196, (2008); Morales C, et al. J Immunol 183(8), 5121-5128, (2009)).

Hereditary forms of open angle glaucoma are caused by mutations in myocilin (MYOC), which lead to a toxic gain of function: protein aggregation and TM cell death. Selective inhibition or gene silencing of Grp94, small molecules capable of reducing the levels of mutant myocilin by selectively inhibiting Grp94, methods for rescuing the IOP phenotype and restores retinal cell health and function are needed. The compositions and methods described herein address these and other needs.

### SUMMARY

Topical and chronic administration of steroids can result in elevated intraocular pressure (IOP) due to steroid-dependent mechanisms. If maintained, this elevated intraocular pressure can result in ocular hypertension and glaucoma, an ocular disorder characterized by progressive damage to the optic nerve and eventually, blindness. Currently, the only treatment option for patients diagnosed with a steroid-induced glaucoma is a cessation of the prescribed steroid regimen. However, some patients present irreversible elevation in intraocular pressure, despite cessation. Though the cause of the changes to intraocular pressure remain unknown, the inventors have established that the biochemical changes are dependent on the activity of Grp94.

The inventors have evidence that Grp94 activity is important for the development of steroid-induced glaucoma and inhibition of Grp94 can ablate the increase in intraocular pressure observed following the administration of steroids to mice. Inhibition of Grp94 did not decrease the intraocular pressure in control animals, demonstrating that Grp94 inhibition is not a regulator of humor outflow or production. The therapeutic can inhibit a protein necessary for the steroid-induced changes in eye biology to occur. The therapeutic can allow for the continued use of prescribed steroids without the risk of eye damage and blindness.

Provided herein are compositions and methods for preventing, treating, or reducing steroid-induced ocular hypertension or glaucoma in a patient. The compositions can be in the form of an ophthalmic solution comprising a therapeutically effective amount of a Grp94-selective inhibitor and a pharmaceutically acceptable vehicle. The Grp94-selective inhibitor can be a compound having a structure represented by Formula II or a pharmaceutically acceptable salt, solvate, derivative, or prodrug thereof:

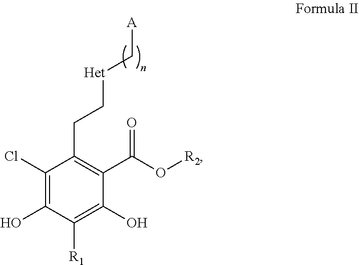

wherein
“Het” is a 5 membered heterocyclic moiety selected from a diazole moiety (such as pyrazole or imidazole) or a triazole moiety (such as 1,2,3-triazole or 1,2,4-triazole);
A is a substituted or unsubstituted C₆-C₁₀ aryl or substituted or unsubstituted C₃-C₁₀ heteroaryl;
R₁ and R2 are independently selected from hydrogen, halogen, C₁-C₆ alkyl, C₁-C₆ alkoxy, C₁-C₆ alkyl ether, C₁-C₆ alkyl halide, C₁-C₆ hydroxyalkyl, C₁-C₆ amino alkyl, C₃-C₆ cycloalkyl, or C₃-C₆ heterocycloalkyl; and
n is an integer from 0 to 5, preferably from 1 to 5, wherein the alkyl group which n defines is substituted or unsubstituted.

In specific examples, the Grp94-selective inhibitor can include 4-Br—BnIm or a derivative thereof.

The methods for preventing, treating, or reducing steroid-induced ocular hypertension or glaucoma in a patient can include administering a composition comprising a therapeutically effective amount of Grp94-selective inhibitor to the patient in need. The composition comprising the Grp94-selective inhibitor can be administered prior, during, or after the patient has been treated with a steroid. In some examples, the methods described herein prevent or reduce steroid-induced ocular hypertension or glaucoma in the patient.

The compositions described herein can be administered orally, intravenously, sublingually, ocularly, topically, transdermally, nasally, or intraperitoneally. Preferably, the compositions are administered transdermally or topically. More preferably, the compositions are administered topically to the eye.

The patient can be a mammal such as a human.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

For a fuller understanding of the disclosure, reference should be made to the following detailed description, taken in connection with the accompanying drawings, in which:

FIGS. 1A-1C show Grp94, but not myocilin, is necessary for SIG. FIG. 1A shows a visual representation of treatment strategy. FIG. 1B is a graph of intraocular pressure (IOP) measurements from wildtype (WT) mice treated with dexamethasone (Dex), 4Br—BnIm, or appropriate vehicles (PBS and PBS with DMSO, respectively). FIG. 1C is a graph of intraocular pressure (IOP) measurements from myocilin knockout (Myoc-KO, KO) mice treated with dexamethasone (Dex), 4Br—BnIm, or appropriate vehicles (PBS and PBS with DMSO, respectively). Measurements are presented as group means from both eyes of each mouse +/−SEM. Group n values are listed in legend as “number of mice/number of eyes.” *P<0.5, **P<0.01, ***P<0.001, and ****P<0.0001, by two-way analysis of variance.

FIGS. 2A-2B show in vivo dexamethasone-induced Collagen I and Fibronectin; Grp94 dependent. FIG. 2A is a representative dot blot of lysates from anterior portion of eyes collected from SIG model WT mice. Samples were run in duplicate for each group; n=4/8 (mice/eyes). FIG. 2B is an analysis of FIG. 2A. Samples plotted as mean±% CV. Student's t-test was used for statistical analysis between individual groups. *P<0.5.

FIGS. 3A-3D show in vitro intracellular and extracellular Grp94-dependent Collagen I and Fibronectin protein levels. FIG. 3A is a western blot from SDS-PAGE separated HTM cell lysates; treated as indicated. Representative blot of duplicated samples. FIG. 3B shows quantitation of FIG. 3A. Samples plotted as mean±% CV. Student's t-test was used for statistical analysis between individual groups. *P<0.5, **P<0.01. FIG. 3C shows a dot blot of cell culture medium from HTM cells treated as indicated. FIG. 3D shows quantitation of FIG. 3C; n=4. Samples plotted as mean±% CV. Student's t-test was used for statistical analysis between individual groups. *P<0.5, **P<0.01, ***P<0.001, and ***P<0.0001.

FIG. 4 is a graph depicting myocilin enhances steroid-induced glaucomatous phenotypes. Absence of myocilin delays the onset of the IOP phenotype by one week; indicating, though not necessary for steroid-induced glaucoma, myocilin contributes to this disorder.

FIG. 5 is an image depicting dexamethasone alters levels of ECM components in vivo; rescued by Grp94 inhibition. Eyes from mice treated as part of IOP experiments were enucleated and lysed. Lysates were probed by dot blot for levels of ECM components.

FIG. 6 is an image of a Western blot demonstrating the dexamethasone alters fibronectin levels in vivo; rescued by Grp94 inhibition. Eyes from mice treated as part of IOP experiments were enucleated and lysed. Lysates were probed by Western blot for levels of Fibronectin.

### DETAILED DESCRIPTION

In the following detailed description, reference is made to the accompanying drawings, which form a part hereof, and within which are shown by way of illustration specific embodiments by which the disclosure can be practiced. It is to be understood that other embodiments by which the disclosure can be practiced. It is to be understood that other embodiments can be utilized and structural changes can be made without departing from the scope of the disclosure.

General Definitions

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supersedes any disclosure of an incorporated publication to the extent there is a contradiction.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. Where particular values are described in the application and claims, unless otherwise stated, the term “about” meaning within an acceptable error range for the particular value should be assumed. In some cases, the term “about” means ±15%.

Concentrations, amounts, solubilities, and other numerical data can be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

As used in the specification and claims, the singular form “a”, “an” and “the” include plural references unless the context clearly dictates otherwise. For example, the term “a nanoparticle” includes a plurality of nanoparticles, including mixtures thereof.

“Patient” is used to describe an animal, preferably a human, to whom treatment is administered, including prophylactic treatment with the compositions of the present disclosure.

The “therapeutically effective amount” for purposes herein is thus determined by such considerations as are known in the art. A therapeutically effective amount of the Grp94 inhibitor is that amount necessary to provide a therapeutically effective result in vivo. The amount of Grp94 inhibitor must be effective to achieve a response, including but not limited to total prevention of (e.g., protection against) and to improved survival rate or more rapid recovery, or improvement or elimination of symptoms associated with eye disorders such as steroid-induced ocular hypertensions and glaucomas, or other indicators as are selected as appropriate measures by those skilled in the art. In accordance with the present disclosure, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of a mammal and the route of administration.

“Administration” or “administering” is used to describe the process in which a small molecule inhibitor such as a Grp94 inhibitor of the present disclosure is delivered to a patient. The composition can be administered in various ways including parenteral (referring to intravenous, intraarterial and other appropriate parenteral routes), intraocular, topically, orally, and percutaneously, among others. Each of these conditions can be readily treated using other administration routes of Grp94 inhibitors to treat a disease or condition.

Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

As used herein, the term “alkyl” refers to straight-chained, branched, or cyclic, saturated hydrocarbon moieties. Unless otherwise specified, C₁-C₂₀ (e.g., C₁-C₁₂, C₁-C₁₀, C₁-C₈, C₁-C₆, C₁-C₄, C₁, C₂, C₃, C₄, C₅, C₆, C₇, C₈) alkyl groups are intended. Examples of alkyl groups include methyl, ethyl, propyl, isopropyl, 1-methyl-ethyl, butyl, isobutyl, t-butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, pentyl, hexyl, heptyl, octyl, nonyl, decyl, dodecyl, tetradecyl, hexadecyl, eicosyl, tetracosyl, and the like. Alkyl substituents can be unsubstituted or substituted with one or more chemical moieties. Examples of suitable substituents include, for example, hydroxy, halogen, nitro, cyano, formyl, C₁C₈ alkyl, C₁C₈ haloalkyl, C₃-C₁₂ cycloalkyl, C₃-C₁₂ heterocycloalkyl, C₂-C₈ alkenyl, C₂-C₈ haloalkenyl, C₃-C₁₂ cycloalkenyl, C₃-C₁₂ heterocycloalkenyl, C₂-C₈ alkynyl, C₁-C₈ alkoxy, C₁-C₈ haloalkoxy, C₁-C₈ alkoxycarbonyl, hydroxycarbonyl, C₁-C₈ acyl, C₁-C₈ alkylcarbonyl, C₆-C₁₀ aryl, C₆-C₁₀ heteroaryl, amino, amido, C₁C₈ carbamoyl, C₁-C₈ halocarbamoyl, phosphonyl, silyl, sulfinyl, C₁-C₆ alkylsulfinyl, C₁-C₆ haloalkylsulfinyl, sulfonyl, C₁-C₆ alkylsulfonyl, C₁-C₆ haloalkylsulfonyl, sulfonamide, thio, C₁-C₆ alkylthio, C₁-C₆ haloalkylthio, C₁-C₆ alkylaminocarbonyl, C₁-C₆ dialkylaminocarbonyl, C₁-C₆ haloalkoxycarbonyl, and haloalkylaminocarbonyl, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e. , C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

As used herein, the term “alkyl halide” or “haloalkyl” refers to straight-chained or branched alkyl groups, wherein these groups the hydrogen atoms can partially or entirely be substituted with halogen atoms. Unless otherwise specified, C₁-C₂₀ (e.g., C₁-C₁₂, C₁-C₁₀, C₁-C_{8,} C₁-C₆, C₁-C₄) alkyl groups are intended. Examples include chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2-difluoroethyl, 2,2-dichloro-2-fluoroethyl, 2,2,2-trichloroethyl, pentafluoroethyl, and 1,1,1-trifluoroprop-2-yl. Haloalkyl substituents can be unsubstituted or substituted with one or more chemical moieties. Examples of suitable substituents include, for example, hydroxy, nitro, cyano, formyl, C₁-C₈ alkyl, C₁-C₈ haloalkyl, C₃-C₁₂ cycloalkyl, C₃-C₁₂ heterocycloalkyl, C₂-C₈ alkenyl, C₂-C₈ haloalkenyl, C₃-C₁₂ cycloalkenyl, C₃-C₁₂ heterocycloalkenyl, C₂-C₈ alkynyl, C alkoxy, C haloalkoxy, C alkoxycarbonyl, hydroxycarbonyl, C₁-C₈ acyl, C₁-C₈ alkylcarbonyl, C₆-C₁₀ aryl, C₆-C₁₀ heteroaryl, amino, amido, C carbamoyl, C halocarbamoyl, phosphonyl, silyl, sulfinyl, C alkylsulfinyl, C haloalkylsulfinyl, sulfonyl, C alkylsulfonyl, C haloalkylsulfonyl, sulfonamide, thio, C₁-C₆ alkylthio, C₁-C₆ haloalkylthio, C₁-C₆ alkylaminocarbonyl, C₁-C₆ dialkylaminocarbonyl, C₁-C₆ haloalkoxycarbonylC₁-C₆ haloalkylcarbonyl, and haloalkylaminocarbonyl, provided that the substituents are sterically compatible and the rules of chemical bonding and strain energy are satisfied.

As used herein, the term “alkoxy” refers to a group of the formula —OZ¹, where Z¹ is unsubstituted or substituted alkyl as defined above. In other words, as used herein an “alkoxy” group is an unsubstituted or substituted alkyl group bound through a single, terminal ether linkage. Unless otherwise specified, alkoxy groups wherein Z¹ is a C₁-C₂₀ (e.g., C₁-C₁₂, C₁-C₁₀, C₁-C₈, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-pentoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-l-methyl-propoxy, and 1-ethyl-2-methyl-propoxy.

As used herein, the term “aryl,” as well as derivative terms such as aryloxy, refers to groups that include a monovalent aromatic carbocyclic group of from 6 to 14 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphtyl, phenylcyclopropyl, and indanyl. In some embodiments, the aryl group can be a phenyl, indanyl or naphthyl group. The term “heteroaryl”, as well as derivative terms such as “heteroaryloxy”, refers to a 5- or 6-membered aromatic ring containing one or more heteroatoms, viz., N, O or S; these heteroaromatic rings can be fused to other aromatic systems. The aryl or heteroaryl substituents can be unsubstituted or substituted with one or more chemical moieties. Examples of suitable substituents include, for example, hydroxy, halogen, nitro, cyano, formyl, C₁-C₈ alkyl, C₁-C₈ haloalkyl, C₃-C₁₂ cycloalkyl, C₃-C₁₂ heterocycloalkyl, C₂-C₈ alkenyl, C₂-C₈ haloalkenyl, C₃-C₁₂ cycloalkenyl, C₃-C₁₂ heterocycloalkenyl, C₂-C₈ alkynyl, C₁-C₈ alkoxy, C₁-C₈ haloalkoxy, C₁-C₈ alkoxycarbonyl, hydroxycarbonyl, C₁-C₈ acyl, C₁-C₈ alkylcarbonyl, C₆-C₁₀ aryl, C₆-C₁₀ heteroaryl, amino, amido, C₁-C₈ carbamoyl, C₁-C₈ halocarbamoyl, phosphonyl, silyl, sulfinyl, C₁-C₆ alkylsulfinyl, C₁-C₆ haloalkylsulfinyl, sulfonyl, C₁-C₆ alkylsulfonyl, C₁-C₆ haloalkylsulfonyl, sulfonamide, thio, C₁-C₆ alkylthio, C₁-C₆ haloalkylthio, C₁-C₆ alkylaminocarbonyl, C₁-C₆ dialkylaminocarbonyl, C₁-C₆ haloalkoxycarbonyl, C₁-C₆ haloalkylcarbonyl, and haloalkylaminocarbonyl, provided that the substituents are sterically compatible and the rules of chemical bonding and strain energy are satisfied.

As used herein, the terms “amine” or “amino” refers to a group of the formula —NZ¹Z², where Z¹ and Z² can independently be a hydrogen, alkyl, haloalkyl, alkenyl, alkynyl, aryl, heteroaryl, cycloalkyl, cycloalkenyl, heterocycloalkyl, or heterocycloalkenyl group as described above. As used herein, the term “alkylamino” refers to an amino group substituted with one or two unsubstituted or substituted alkyl groups, which can be the same or different. As used herein, the term “haloalkylamino” refers to an alkylamino group wherein the alkyl carbon atoms are partially or entirely substituted with halogen atoms.

As used herein, the term “phosphate-containing” group refers to a salt or ester of an oxygen acid of phosphorus or a phosphorus oxo acid. In its salt form or base form, a phosphate-containing group contains at least one metal ion or an ammonium ion. The phosphate containing group includes partial and complete esters of phosphorus oxo acids. Examples of phosphate-containing groups include a phosphinic acid, phosphonic acid, phosphoric acid, pyrophosphoric phosphinic acid, pyrophosphoric acid groups, partial and complete esters and salts thereof. For example, the phosphate-containing group includes “phosphonyl” which refers to a group of the formula

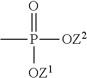

where Z¹ and Z² can independently be a hydrogen, C₁C₈ alkyl, C₁-C₈ haloalkyl, C₂-C₈ alkenyl, C₂-C₈ alkynyl, C₆-C₁₀ aryl, C₆-C₁₀ heteroaryl, C₃-C₁₂ cycloalkyl, C₃-C₁₂ cycloalkenyl, C₃-C₁₂ heterocycloalkyl, or C₃-C₁₂ heterocycloalkenyl group as described above. As used herein “alkylphosphonyl” refers to a phosphonyl group substituted with one or two unsubstituted or substituted alkyl groups, which can be the same or different. As used herein, the term “haloalkylphosphonyl” refers to an alkylphosphonyl group wherein the alkyl carbon atoms are partially or entirely substituted with halogen atoms.

As used herein, Me refers to a methyl group; OMe refers to a methoxy group; and i-Pr refers to an isopropyl group.

As used herein, the term “halogen” including derivative terms such as “halo” refers to fluorine, chlorine, bromine and iodine.

The term “hydroxyl” as used herein is represented by the formula —OH.

The term “nitro” as used herein is represented by the formula —NO₂.
The term “cyano” as used herein is represented by the formula —CN.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A prodrug refers to a compound that is made more active in vivo. Certain compounds disclosed herein can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they can be easier to administer than the compound, or parent drug. They can, for instance, be bioavailable by oral administration whereas the parent drug is not. The prodrug can also have improved solubility in pharmaceutical compositions over the parent drug. A wide variety of prodrug derivatives are known in the art, such as those that rely on hydrolytic cleavage or oxidative activation of the prodrug.

Prodrugs of any of the disclosed compounds include, but are not limited to, carboxylate esters, carbonate esters, hemi-esters, phosphorus esters, nitro esters, sulfate esters, sulfoxides, amides, carbamates, azo compounds, phosphamides, glycosides, ethers, acetals, and ketals. Oligopeptide modifications and biodegradable polymer derivatives (as described, for example, in Int. J. Pharm. 115, 61-67, 1995) are within the scope of the present disclosure. Methods for selecting and preparing suitable prodrugs are provided, for example, in the following: T. Higuchi and V. Stella, “Prodrugs as Novel Delivery Systems,” Vol. 14, ACS Symposium Series, 1975; H. Bundgaard, Design of Prodrugs, Elsevier, 1985; and Bioreversible Carriers in Drug Design, ed. Edward Roche, American Pharmaceutical Association and Pergamon Press, 1987.

Reference will now be made in detail to specific aspects of the disclosed materials, compounds, compositions, articles, and methods, examples of which are illustrated in the accompanying Examples and Drawings.

Compounds and Compositions

The compounds used in the present disclosure and in the pharmaceutical compositions disclosed herein can be administered individually, or in combination with or concurrently with one or more other compounds used in other embodiments of the present disclosure. Additionally, the compounds used in the present disclosure can be administered in combination with or concurrently with other therapeutics for glaucoma disorders.

The compounds can include any Grp94-selective inhibitor. Grp94-selective inhibitors are known in the art and are described for example, in U.S. Pat. No. 8,685,966 to Blagg et al., which is incorporated herein by reference in its entirety.

In some embodiments, the Grp94-selective inhibitor can include a compound having a structure represented by Formula I or a pharmaceutically acceptable salt, solvate, derivative, or prodrug thereof:

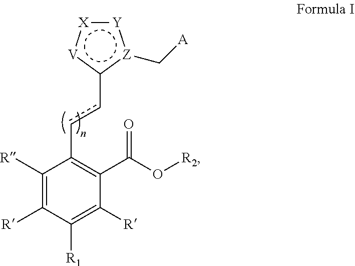

wherein,
V, X, and Y are each independently selected from CH, NH or N;
Z is selected from C or N;
R₁ and R2 are independently selected from hydrogen, halogen, C₁-C₆ alkyl, C₁-C₆ alkoxy, C₁-C₆ alkyl ether, C₁-C₆ alkyl halide, C₁-C₆ hydroxyalkyl, C₁-C₆ amino alkyl, C₃-C₆ cycloalkyl, or C₃-C₆ heterocycloalkyl;
R′, for each occurrence, is independently selected from the group consisting of H. OH, SH, and NH₂;
R″ is selected from the group consisting of hydrogen, halogen, C₁-C₆ alkyl, C₁-C₆ alkoxy, C₁-C₆ alkyl ether, C₁-C₆ alkyl halide, C₁-C₆ hydroxyalkyl, C₁-C₆ amino alkyl, C₃-C₆ cycloalkyl, or C₃-C₆ heterocycloalkyl;
A is selected from the group consisting of substituted or unsubstituted aryl, substituted or unsubstituted cycloalkyl, substituted or unsubstituted heterocycloalkyl, or substituted or unsubstituted heteroaryl; and
n is 0 to 5, preferably an integer from 1 to 5, wherein the alkyl group which n defines is substituted or unsubstituted.

In certain embodiments, the Grp94-selective inhibitor can include a compound having a structure represented by Formula II or a pharmaceutically acceptable salt, solvate, derivative, or prodrug thereof:

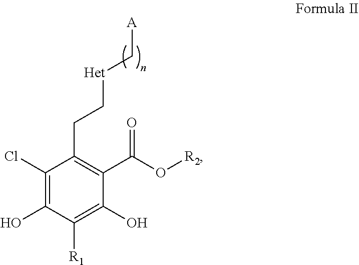

wherein
“Het” is a heterocyclic moiety;
A is a substituted or unsubstituted aryl or substituted or unsubstituted heteroaryl;
R₁ and R2 are independently selected from hydrogen, halogen, C₁-C₆ alkyl, C₁-C₆ alkoxy, C₁-C₆ alkyl ether, C₁-C₆ alkyl halide, C₁-C₆ hydroxyalkyl, C₁-C₆ amino alkyl, C₃-C₆ cycloalkyl, or C₃-C₆ heterocycloalkyl; and
n is from 0 to 5, preferably an integer from 1 to 5, wherein the alkyl group which n defines is substituted or unsubstituted.

In some aspects of the Formulas disclosed herein, “Het” is a heterocycle selected from a single or multi-cyclic aromatic or non-aromatic ring systems in which at least one of the ring members is an element other than carbon. Heterocycle compounds can include, but are not limited to, pyridine, pyrimidine, furan, thiopene, pyrrole, isoxazole, isothiozole, pyrazole, oxazole, thiazole, imidazole, oxadiazole, thiadiazole, triazole, pyridazine, pyrimidine, pyrazine, triazine, tetrazine, pyrrolidine, piperidine, piperazine, morpholine, azetidine, tetrahydrophan, tetrahydrofuran, dioxane and the like. Preferably, the heterocycles used in the present disclosure should exhibit a conformational bias for the cis-amide conformation in order to project the hydrophobic aromatic compound into the Grp94 hydrophobic pocket.

In some aspects of Formula I or II, A can be a substituted C₆-C₁₀ aryl, an unsubstituted C₆-C₁₀ aryl, a substituted C₃-C₁₀ heteroaryl, or an unsubstituted C₃-C₁₀ heteroaryl. Examples of aryl and heteroaryl groups are described herein.

In some aspects of Formula I or II, A can be substituted. Examples of suitable substituents include, for example, hydroxy, halogen (such as fluoro, chloro, or bromo), nitro, cyano, formyl, C₁-C₈ alkyl, C₁-C₈ haloalkyl, C₃-C₁₂ cycloalkyl, C₃-C₁₂ heterocycloalkyl, C₂-C₈ alkenyl, C₂-C₈ haloalkenyl, C₃-C₁₂ cycloalkenyl, C₃-C₁₂ heterocycloalkenyl, C₂-C₈ alkynyl, C₁-C₈ alkoxy, C₁-C₈ haloalkoxy, C₁-C₈ alkoxycarbonyl, hydroxycarbonyl, C₁-C₈ acyl, C₁-C₈ alkylcarbonyl, C₆-C₁₀ aryl, C₆-C₁₀ heteroaryl, amino, amido, C₁-C₈ carbamoyl, C₁-C₈ halocarbamoyl, phosphonyl, silyl, sulfinyl, C₁-C₆ alkylsulfinyl, C₁-C₆ haloalkylsulfinyl, sulfonyl, C₁-C₆ alkylsulfonyl, C₁-C₆ haloalkylsulfonyl, sulfonamide, thio, C₁-C₆ alkylthio, C₁-C₆ haloalkylthio, C₁-C₆ alkylaminocarbonyl, C₁-C₆ dialkylaminocarbonyl, C₁-C₆ haloalkoxycarbonyl, or haloalkylaminocarbonyl.

In certain embodiments, the Grp94-selective inhibitor can include a compound having a structure represented by Formula I, II, or a pharmaceutically acceptable salt, solvate, derivative, or prodrug thereof wherein “Het” is a 5 membered heterocyclic moiety selected from a diazole moiety (such as pyrazole or imidazole) or a triazole moiety (such as 1,2,3-triazole or 1,2,4-triazole); A is a substituted or unsubstituted C₆-C₁₀ aryl or substituted or unsubstituted C₃-C₁₀ heteroaryl; R₁ and R₂ are independently selected from hydrogen, halogen, C₁-C₆ alkyl, C₁-C₆ alkoxy, C₁-C₆ alkyl ether, C₁-C₆ alkyl halide, C₁-C₆ hydroxyalkyl, C₁-C₆ amino alkyl, C₃-C₆ cycloalkyl, or C₃-C₆ heterocycloalkyl; and n is from 0 to 5, preferably an integer from 1 to 5.

In some embodiments of Formula I or II, A can be a substituted or unsubstituted C₆ aryl or a substituted or unsubstituted C₃-C₆ heteroaryl. In some examples of Formula I or II, A can be a substituted C₆ aryl or a substituted C₃-C₆ heteroaryl. The heteroatom in the heteroaryl can be selected from N, S, or O, preferably N or O. The substituent on the aryl or heteroaryl can include a halogen such as a fluoro, a chloro, a bromo, or an iodo group.

In certain embodiments, the Grp94-selective inhibitor can include a compound having a structure represented by Formula IIA:

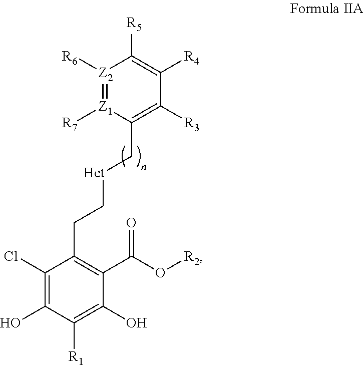

wherein
R₁ and R₄ are independently selected from hydrogen, halogen, C₁-C₃ alkyl, preferably both R₁ and R₄ are hydrogen;
R₂ is C₁-C₆ alkyl, preferably methyl, ethyl, or propyl;
R₃ is hydrogen, halogen, hydroxyl, amino, cyano, nitro, C₁-C₆ alkyl, C₁-C₆ alkoxy, C₁-C₆ hydroxyalkyl, or C₁-C₆ aminoalkyl;
R₅ is halogen, C₁-C₆ alkyl, C₂-C₆ alkenyl, C₂-C₆ alkynyl, C₁-C₆ alkoxy, C₁-C₆ alkyl halide, hydroxyl, amino, cyano, or nitro;
R₆ and R₇ are independently present or absent and are independently selected from hydrogen, halogen, hydroxy, C₁-C₆ alkyl, C₁-C₆ alkoxy, C₁-C₆ alkyl halide, C₁-C₆ hydroxyalkyl, or C₁-C₆ aminoalkyl; and
Z₁ and Z₂ are independently selected from C or N.

In certain embodiments, the Grp94-selective inhibitor can include a compound having a structure represented by Formula IIA-1:

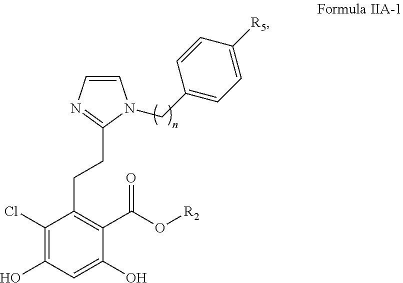

wherein
R2 is C₁-C₆ alkyl, preferably methyl, ethyl, or propyl;
R₅ is halogen, preferably chloro or bromo; and
n is 0, 1, 2, 3 or 4.

In some embodiments of Formula I, II, IIA, or IIA-1, n can be an integer selected from 1, 2, or 3. In some examples, n is 1.

In some embodiments of Formula I, II, IIA, or IIA-1, R₂ can be methyl or ethyl. R₂ is preferably methyl.

In some embodiments of Formula I, II, IIA, or IIA-1, R₅ can be a halogen. R₅ is preferably chloro or bromo. R₅ is more preferably bromo.

In some embodiments of Formula I, II, IIA, or IIA-1, the Grp94-selective inhibitor is methyl 2-(2-(1(4-bromobenzyl)-1H-imidazol-2-yl)ethyl)-3-chloro-4,6-dihydroxybenzoate (4-Br—BnIm) or a derivative thereof. In particular, the Grp94-selective inhibitor can have the structure:

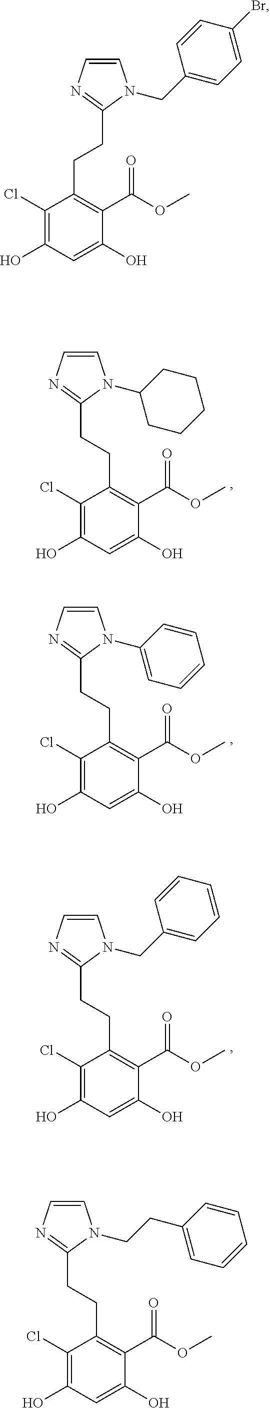 [SMILES: COC(=O)c1c(O)cc(O)c(Cl)c1CCc1nccn1Cc1ccc(Br)cc1 ; COC(=O)c1c(O)cc(O)c(Cl)c1CCc1nccn1C1CCCCC1 ; COC(=O)c1c(O)cc(O)c(Cl)c1CCc1nccn1-c1ccccc1 ; COC(=O)c1c(O)cc(O)c(Cl)c1CCc1nccn1Cc1ccccc1 ; COC(=O)c1c(O)cc(O)c(Cl)c1CCc1nccn1CCc1ccccc1]

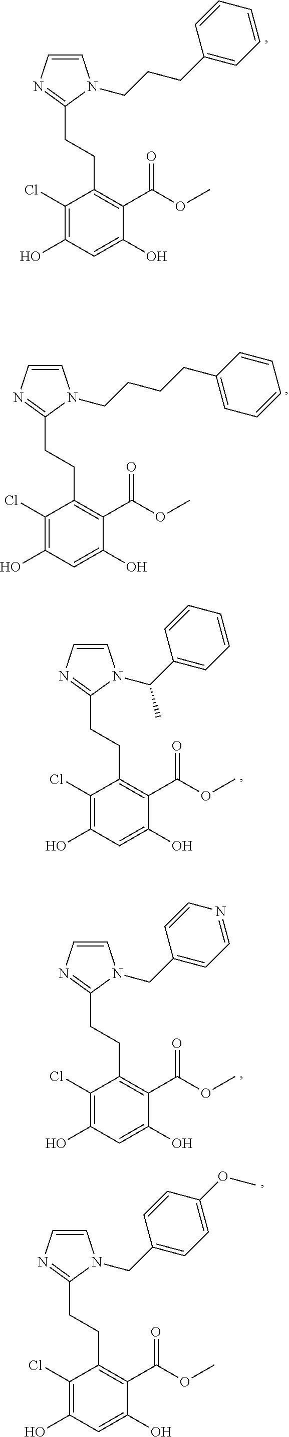 [SMILES: COC(=O)c1c(O)cc(O)c(Cl)c1CCc1nccn1CCCc1ccccc1 ; COC(=O)c1c(O)cc(O)c(Cl)c1CCc1nccn1CCCCc1ccccc1 ; COC(=O)c1c(O)cc(O)c(Cl)c1CCc1nccn1[C@@H](C)c1ccccc1 ; COC(=O)c1c(O)cc(O)c(Cl)c1CCc1nccn1Cc1ccncc1 ; COC(=O)c1c(O)cc(O)c(Cl)c1CCc1nccn1Cc1ccc(OC)cc1]

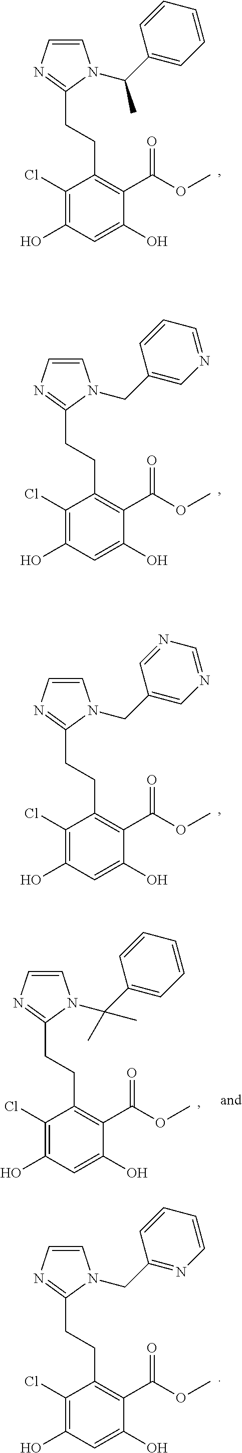 [SMILES: COC(=O)c1c(O)cc(O)c(Cl)c1CCc1nccn1[C@H](C)c1ccccc1 ; COC(=O)c1c(O)cc(O)c(Cl)c1CCc1nccn1Cc1cccnc1 ; COC(=O)c1c(O)cc(O)c(Cl)c1CCc1nccn1Cc1cncnc1 ; COC(=O)c1c(O)cc(O)c(Cl)c1CCc1nccn1C(C)(C)c1ccccc1 ; COC(=O)c1c(O)cc(O)c(Cl)c1CCc1nccn1Cc1ccccn1]

Pharmaceutical Compositions

The pharmaceutical compositions disclosed herein can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the disclosure. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton, Pa., Mack Publishing Company, 19^{th} ed.) describes formulations which can be used in connection with the subject disclosure.

For ease of administration, the disclosed compounds can be formulated into various pharmaceutical forms. As appropriate compositions, there can be cited all compositions usually employed for systemically or topically administering drugs. These pharmaceutical compositions are desirably in unitary dosage form suitable, preferably, for administration orally, topically, percutaneously, or by parenteral injection. For example, in preparing the compositions in oral dosage form, any of the usual pharmaceutical media can be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules often represent the most advantageous oral dosage unit form, in which case solid pharmaceutical carriers are obviously employed. For parenteral compositions, the carrier will usually comprise sterile water, at least in large part, though other ingredients, for example, to aid solubility, can be included. Injectable solutions, for example, can be prepared in which the carrier comprises saline solution, glucose solution or a mixture of saline and glucose solution. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives can facilitate the administration to the skin and/or can be helpful for preparing the desired compositions.

In some embodiments, the pharmaceutical compositions can be formulated as an ophthalmic solution. The ophthalmic solution can comprise a therapeutically effective amount of a Grp94-selective inhibitor and a pharmaceutically acceptable vehicle. The ophthalmic solution can include from 0.1 to 10% by weight of the Grp94-selective inhibitor, based on the weight of the ophthalmic solution. For example, the ophthalmic solution can include from 0.1 to 10% by weight of 4Br—BnIm, based on the weight of the ophthalmic solution.

The amount of the compound in the drug composition will depend on absorption, distribution, metabolism, and excretion rates of the drug as well as other factors known to those of skill in the art. Dosage values can also vary with the severity of the condition to be alleviated. The compounds can be administered once or can be divided and administered over intervals of time. It is to be understood that administration can be adjusted according to individual need and professional judgment of a person administrating or supervising the administration of the compounds used in the present disclosure.

The dose of the compounds administered to a subject can vary with the particular composition, the method of administration, and the particular disorder being treated. The dose should be sufficient to affect a desirable response, such as a therapeutic or prophylactic response against a particular disorder or condition. It is contemplated that one of ordinary skill in the art can determine and administer the appropriate dosage of compounds disclosed in the current disclosure according to the foregoing considerations.

Dosing frequency for the composition includes, but is not limited to, at least about once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day. In some embodiments, the interval between each administration is constant. For example, the administration can be carried out daily, every two days, every three days, every four days, every five days, or weekly. In some embodiments, the administration can be carried out twice daily, three times daily, or more frequent. Administration can also be continuous and adjusted to maintaining a level of the compound within any desired and specified range.

The administration of the composition can be extended over an extended period of time, such as from about a month or shorter up to about three years or longer. For example, the dosing regimen can be extended over a period of any of about 2, 3, 4, 5, 6, 7, 8, 9, 10, 11, 12, 18, 24, 30, and 36 months. In some embodiments, there is no break in the dosing schedule. In some embodiments, the interval between each administration is no more than about a week.

Methods

The data described herein demonstrate a role for Grp94 in the generation of secondary forms of glaucoma that develop following the administration of steroids, such as the glucocorticoid (GC), dexamethasone (dex). The development of endoplasmic reticulum (ER) stress, the induction of WT myocilin expression, and the aberrant regulation of the protein components of the extracellular matrix (ECM) have been implicated in disease progression leading to the inventors' discovery that Grp94 stabilizes aberrant forms of extracellular matrix (ECM) components, ultimately leading to a dysfunctional trabecular meshwork (TM) and glaucoma.

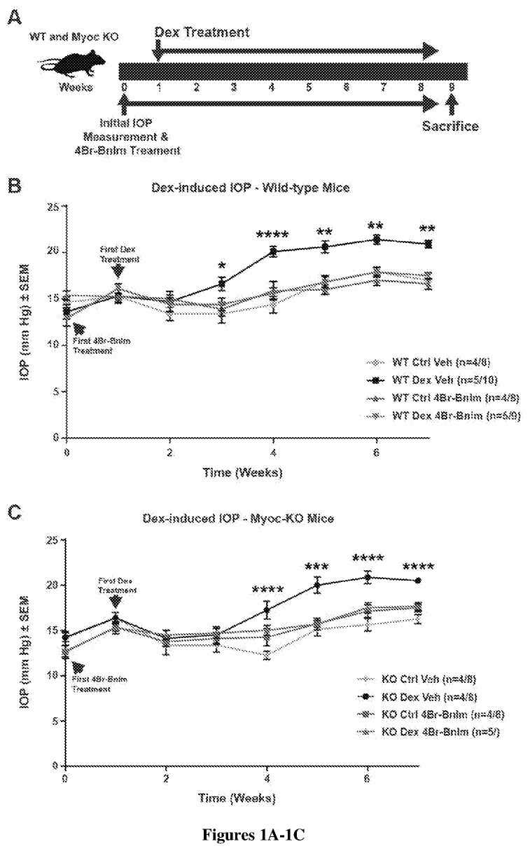

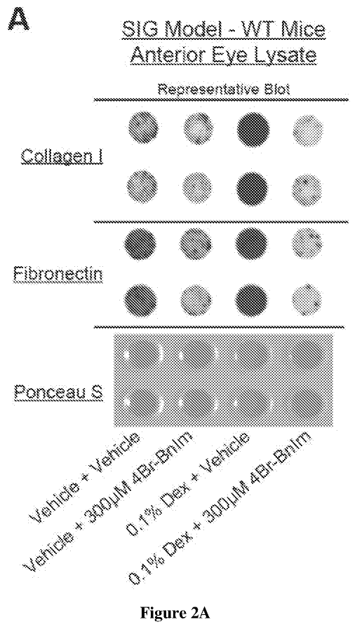

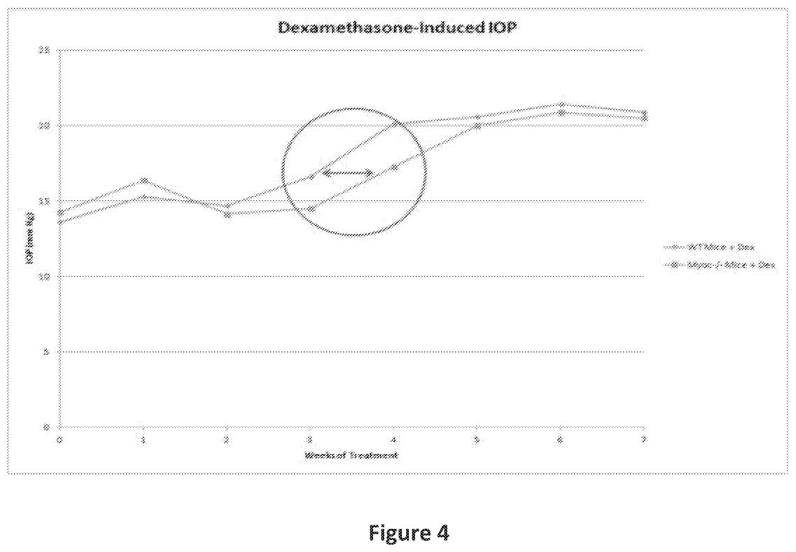

More specifically, the data described herein demonstrates that Grp94 is necessary for the development of steroid-induced glaucoma. WT mice treated with dex eye drops developed elevated intraocular pressure (IOP), whereas WT mice treated with a selective small molecule Grp94 inhibitor prior to treatment with dex did not develop the IOP phenotype (FIG. 4). Myoc-KO mice also develop elevated IOP following dex (FIG. 4), suggesting this form of glaucoma acts independent of myocilin. However, the onset of the IOP phenotype was delayed relative to WT mice (FIG. 4). This data suggests that myocilin can facilitates, but is not necessary for, the development of steroid-induced glaucoma phenotypes. Additionally, inhibition of Grp94 prior to dexamethasone administration ablated the development of the IOP phenotype (FIG. 1C) in the Myoc-KO mice, which demonstrates that Grp94 is necessary for the development and progression of glaucoma independent of myocilin.

Provided herein are methods of preventing, reducing, or treating steroid induced ocular hypertension or glaucoma. In some examples, the methods include preventing or reducing steroid-induced ocular hypertension prior to administration of a steroid. In some examples, the methods include preventing or reducing steroid-induced glaucoma prior to administration of a steroid. These methods can include administering a Grp94-selective inhibitor, as described herein, to the patient. In particular, the methods can include administering to the patient a therapeutically effective amount of one or more Grp94-selective inhibitor or a pharmaceutically acceptable salt, ester, derivative or prodrug thereof, or compositions thereof.

As the methods described herein can prevent or reduce the occurrence of steroid induced glaucoma, the Grp94-selective inhibitor or composition can be administered prior to or during administration of a steroid.

The compounds and compositions can be adapted for topical such as ocular, oral, rectal, vaginal, parenteral, intramuscular, intraperitoneal, intraarterial, intrathecal, intrabronchial, subcutaneous, intradermal, intravenous, nasal, buccal or sublingual routes of administration. For ocular or topical delivery, the composition can be in the form of a solution or emulsion which are in a form suitable for example, eye drops, gels, ointments, sprays, creams or specialist ocular delivery devices. For oral administration, particular use can be made of compressed tablets, pills, tablets, gellules, drops, and capsules. An alternative means is by transdermal administration, for example by use of a skin patch. For example, the active ingredient can be incorporated into a cream consisting of an aqueous emulsion of polyethylene glycols or liquid paraffin. Other forms of administration comprise solutions or emulsions which can be injected intravenously, intraarterially, intrathecally, subcutaneously, intradermally, intraperitoneally or intramuscularly, and which are prepared from sterile or sterilizable solutions.

In some examples, the methods disclosed herein can include topically administering an amount of a Grp94-selective inhibitor or composition sufficient to prevent, reduce, or treat steroid induced glaucoma or steroid induced ocular hypertension. In specific examples, the methods disclosed herein can include topically administering an amount of a Grp94-selective inhibitor or composition to the eye.

The patient can be a mammal such as a human.

### EXAMPLES

Example 1

### Development of Grp94 Inhibitors with Greater Efficacy and Biological Activity

Described in this example are methods for the development of GRP94 inhibitors with greater efficacy and biological activity by improving the first generation Grp94 inhibitors to produce more potent small molecules with improved solubility and stability and test their selectivity for Grp94 over other Hsp90 isoforms. Compounds can be prioritized based on potency of mutant myocilin degradation in a cell model and pharmacokinetics/pharmacodynamics in mouse ocular tissues. Pharmacodynamic and pharmacokinetic evaluations of Grp94 inhibitors can be performed to advance lead compounds to pre-clinical development. The benchmark compound used for the studies is 4-Br—BnIm

The retention time of these compounds can be established in various eye regions to compare the pharmacokinetics of novel compounds to the lead compound 4Br—BnIm

Method for assessing effects of derived inhibitors on mutant myocilin clearance in vitro: Two HEK-293T-Rex stable cell lines expressing an inducible mutant myocilin (I477N and Y437H) can be treated with various concentrations of DMSO, 4Br—BnIm, or a generated analogue for 24 hours. Cells are then collected and lysed. Intracellular levels of mutant myocilin can be assessed by Western blot. Levels of secreted mutant myocilin can be assessed by dot blot.

Method for assessing ocular pharmacokinetics and pharmacodynamics of novel Grp94 inhibitors: WT and transgenic mice overexpressing the Y437H mutant human myocilin (Tg-MYOCY437H) can receive a topical administration of 3 and 300 μM concentrations of a selective Grp94 inhibitor to each eye. Eyes can be collected at 1, 8, 16, 24, and 48 hours after compound administration. One eye from the Tg-MYOCY437H mice can then be frozen to assess compound pharmacodynamics, as determined by changes in myocilin levels. One eye can be bisected into anterior and posterior regions. Extracts from each region are analyzed for amount of 4-Br—BnIm or test article retained at these time point using HPLC.

### Example 2

### Examination of Inhibitor Selectivity and Role of Glaucoma Pathogenesis Using Structure-Activity Approaches

The inhibitor selectivity and the role of glaucoma pathogenesis using structure-activity approaches can be evaluated. Structure-guided approaches combined with biochemical and cell biology methods identify critical residues in Grp94 that are necessary for imparting Hsp90-isoform selectivity. Selectivity of Grp94 inhibitors can be determined using in vitro systems, as well as structural tools, to create a structure-function profile. In vitro myocilin aggregation kinetics with Grp94 modulators can be characterized. Efficacy of Grp94 inhibitors in Grp94-/- fibroblasts can be assessed to support the preclinical development of this new class of compounds. In summary, elucidation of how Grp94 interacts with myocilin and ECM components using biophysical, analytical, and structural techniques can be accomplished.

Crystal structure of Grp94 N-terminal domain in complex with new compounds: Crystal structures of Grp94 in complex with inhibitors, revealing binding modes that explain selectivity for Grp94 have been previously solved. For example, the truncated N-terminal domain of Canis lupus familiaris Grp94 (NΔ41, cloned by ATUM), which comprises amino acid residues 69-337 but 287-327 is replaced with 4×Gly as in Dollins, D. E., et al., Mol. Cell. 28(1), 41-56, (2007), is expressed and purified as described in Crowley, V. M., et al., J. Med. Chem., 59(7), 3471-3488, (2016)). Purified NΔ41 can be exchanged into 100 mM bicine buffer at pH 7.8 and concentrated to 30 mg mL⁻¹. Crystals grow in a condition containing ˜30% PEG400, 5% glycerol, 100 mM bicine pH 7.8, and 75 mM MgCl₂ and appear within 1 week. Crystals can then be soaked for 20 mM-2d with 1-˜3.5 mM compounds prepared as 50 mM stock solutions in DMSO. For each compound, soaking conditions can be optimized for compound occupancy in the resultant structure. After soaking, crystals can be dragged through 100% glycerol layered atop soaking solutions (final concentration ˜25% in drop) and immediately cryo-cooled in liquid nitrogen. Diffraction data can be collected and processed using HKL-3000 and solved by molecular replacement using as the search model in Phaser the polypeptide chain A of PDB code 2GFD or other similar structures. Models can be iteratively built and refined employing Coot and Phenix Refine, respectively. Models and restraints for compounds can be prepared using eLBOW in Phenix refine using as input SMILES strings generated in ChemDraw.

In vitro myocilin aggregation kinetics with Grp94 modulators: An in vitro assay utilizing purified proteins that is complementary to cellular studies demonstrating that inhibition of Grp94 by 4-Br—BnIm results in degradation of toxic mutant myocilin have been developed. The presence of Grp94 under conditions which the myocilin OLF domain aggregates accelerates aggregation in vitro, and co-aggregation in the end point assay have been observed. Addition of a Grp94 inhibitor (e.g. 4-Br—BnIm) or others rescues Grp94 from co-aggregation with myocilin, indicating the aberrant interaction was shown to be inhibited. The assay has been converted to HTS format (half-height 384-well plates).

Validation of the selectivity of Grp94-selective inhibitors using Grp94-/- fibroblasts: Fibroblast cells treated with 4Br—BnIm and analogues can be used to determine whether mutant myocilin is cleared by these inhibitors when Grp94 is absent.

Culture of Grp94-/- MEFs: MEFs can be cultured on gelatin-coated culture dishes in DMEM containing glucose and fetal bovine serum. For feeder cultures, cells can be plated at 2.5 x 104 cells/cm² and used within 3 days of plating. All gelatin-coated culture dishes are preferably prepared no more than 1 week prior to use.

Transduction of Grp94-/- MEFs with mutant myocilin lentiviral particles: Vectors encoding viral packing proteins (pPAX2, pVSVG, and pLEX containing FLAG-tagged mutant myocilin; I477N or Y437H) can be transfected into HEK293T cells. After 4 hours, the transfection complex is replaced with serum free media and incubated an additional 72 hours. Media can be collected at two intervals, pooled, centrifuged, and filtered. Grp94-/- and wildtype MEFs can be transduced by diluting virus-containing media in fresh serum free. After 6 hours, this solution is replaced with complete media and cells are incubated for 24-72 hours.

Assessment of mutant myocilin reductions by Grp94 inhibition in Grp94-/- MEFs: Using standard Western blot analysis, the effects of Grp94 selective inhibitors on the levels of mutant myocilin can be assessed in Grp94-/- and wildtype MEFs. The levels of mutant myocilin is preferably normalized to GAPDH.

### Example 3

### Evaluation of Biological Efficacy of Grp94 Inhibition Toward Multiple Models of Glaucoma

The biological efficacy of Grp94 inhibition toward multiple models of glaucoma can be evaluated. In particular, the pathways and cellular components necessary for myocilin degradation following Grp94 inhibition have been identified and these findings can be expanded to better understand the mechanisms driving the pathobiology of glaucoma and the role of Grp94 in these processes. Mechanistic studies into the regulation of the ECM and myocilin by Grp94 can be conducted using in vitro, in vivo, and TM cell models. Novel Grp94 selective inhibitors can be validated in a mouse model of steroid-induced glaucoma.

Grp94 stabilization of mutant forms of myocilin which are causative for juvenile open-angle glaucoma can be established. In particular, selective inhibition of Grp94 reduces the intracellular accumulation of mutant myocilin as well as rescue the intraocular pressure phenotype of transgenic mice expressing a human mutant myocilin. The mechanism through which inhibition of Grp94 clears mutant myocilin can be elucidated. These studies involve cell culture manipulations (overexpression and silencing studied) to identify the a) degradation pathway that is responsible for the clearance of mutant myocilin following Grp94 inhibition, and b) the components of the ER molecular chaperone repertoire necessary for myocilin to be cleared.

(iii) Validate degradation mechanism of mutant myocilin following Grp94 inhibition: The inducible mutant myocilin cell lines (see (i) and (ii)) can be treated with established inhibitors of autophagy or proteasomal degradation; specifically: 50 μM Bortezomib or 1 μM Epoxomycin to inhibit proteasomal degradation, or 1 mM 3-methyladenine, 20 μM LY194002, 200 nM Bafilomycin A1, 20 μM Leupeptin, 3 μM Brefeldin A, 50 μM Chloroquine as inhibitors of distinct components of the autophagic protein degradation machinery.

(iv) Myocilin solubility: The inducible mutant myocilin cell lines (see (i) and (ii)), as well as HEK-293T-Rex cells expressing an inducible WT myocilin, can be induced by the addition of tetracycline to the culture medium. GFP (control), WT PPIB, or a prolyl-isomerase null mutant PPIB (R95A) can be transfected in using Lipofectamine 2000. Following 48 hours of transfections, the cells can then be treated with DMSO, 4Br—BnIm, or an analogue, for 24 hours. Cells can then be collected and lysed in lysis buffer with 0.1% Triton-X 100 (a non-ionic detergent). The resulting lysate is centrifuged for 10 minutes at 10,000×gravity. The resulting supernatant and pellet can be separated by SDS-Page and probed for myocilin levels by western blot to detect the levels of soluble and Triton-X insoluble myocilin.

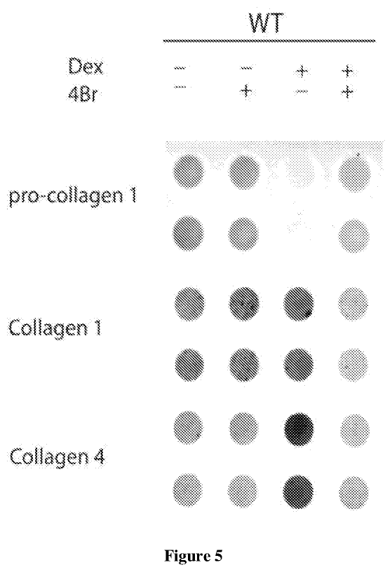

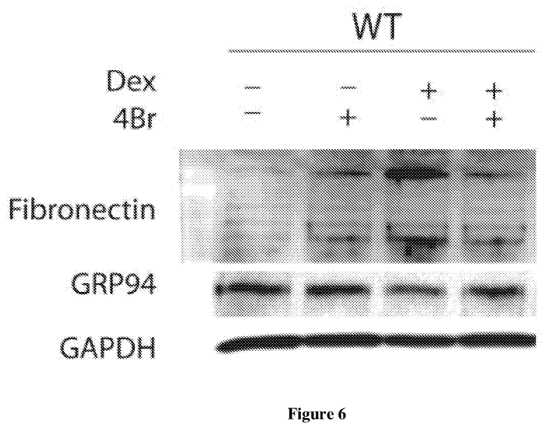

Previous findings in myocilin-driven glaucoma can be expanded to encompass steroid-induced glaucoma. Administration of steroids have been linked to the altered expression of myocilin as well as the expression and physical properties of ECM components such as collagen and fibronectin. Alteration of ECM components have been linked to ER stress and increased cellular rigidity. Additionally, the ER chaperone PPIB, which have been shown to regulate myocilin proteostasis, is a known regulator of ECM components such as collagen. Provided herein are data demonstrating that inhibition of Grp94 prior to topical administration of the glucocorticoid dexamethasone ablates the elevated IOP phenotype associated with steroid regimens. Having identified Grp94 activity as necessary for the development of these pathologies, additional mouse and human trabecular meshwork cell models for mechanistic insight into the pathogenesis of these forms of glaucoma by identifying the ECM components regulated by Grp94 which are altered following dexamethasone administration can be examined The data herein demonstrates that topical administration of dexamethasone to mouse eyes decreases levels of pro-collagen and increases levels of Collagen I, Collagen IV, and Fibronectin. These changes are linked to Grp94 activity as evidenced by the rescue of the aforementioned phenotypes following inhibition of Grp94 (FIGS. 5 and 6). Characterization of the phenotypic outcomes of Grp94 inhibition under the conditions of dexamethasone treatment can be carried out by the distribution and localization of ECM components and cellular rigidity in cell culture models of dex-induced glaucoma, as well as IOP in dexamethasone treated mice.

Role of Grp94 in steroid-induced glaucoma: the role of Grp94 can be assessed by (i) pretreatment with Grp94-selective inhibitor prior to administration of dexamethasone to HTM cell. Cultured human trabecular meshwork (HTM) cells can be used to validate the preliminary in vivo results as follows. HTM cells are treated with DMSO, 4Br—BnIm, or an analogue for 24 or 48 hours. Cell culture media is removed and replaced with culture medium containing 100nM dexamethasone or a control. Cells are cultured for 5 and 10 days. The inventors assess the maturity of the ECM using 1% deoxycholate to disrupt soluble immature ECM, as previously described (Filla, M. S., et al., Exp Eye Res, 165, 7-19, (2017)). Measurements are conducted using fluorescence microscopy. The rigidity of HTM cells under these conditions are assessed using atomic force microscopy as previously described (Raghunathan, V. K., et al., Invest Ophthalmol Vis Sci, 56(8), 4447-59, (2015)). The immunoprecipitate ECM components (Collagen IV, Collagen I, and Fibronectin) can be used to evaluate the chaperone repertoire associating with the ECM under the conditions of dexamethasone, including Grp94, PPIB, and Hsp47. The intracellular and extracellular localization of Grp94, Collagen IV, Collagen I, and Fibronectin is assessed using fluorescent microscopy. Analyses of co-localization between Grp94 and Collagen IV, Collagen I, or Fibronectin is assessed.

The role of Grp94 can also be assessed by (ii) Post-treatment of dexamethasone treated HTM cells with a selective Grp94 inhibitor: HTM cells are treated for 5 or 10 days with 100nM dexamethasone or a control. 24 or 48 hours prior to harvest, dexamethasone or control cells can be treated with DMSO, 4Br—BnIm, or an analogue. Experiments can be performed as in (i).

The role of Grp94 can further be assessed by (iii) Identify alterations in the Grp94-ECM component repertoire following dexamethasone treatment using LC-MS/MS. FLAG-tagged Grp94 can be transduced by lentiviral particles into cultured human trabecular meshwork (HTM) cells. Cells can be cultured for 10 days to ensure expression of FLAG-Grp94. Transduced HTM cells can be treated with DMSO or 4Br—BnIm for 24 hours. Treated HTM cells can be lysed and Grp94-complexes precipitated using anti-FLAG beads. The samples can be separated using SDS-PAGE. Bands identified by Coomassie staining are digested and analyzed by liquid chromatography tandem mass spectroscopy (LC-MS/MS).

The role of Grp94 can also be assessed by (iv) Assess in vivo efficacy of novel selective-Grp94 inhibitors in steroid-induced model of glaucoma. 4-month old C57B6 mice (n=10/group) receive a single topical administration of 300 μM of a 4Br—BnIm analogue, generated as described previously, daily for 7 days. Mice can then receive an additional topical administration of 0.1% dexamethasone sodium phosphate formulated for ocular delivery (Bausch & Lomb) or a control. IOP can be measured once per week. Mice can be anesthetized with isoflurane and measurements are taken using a handheld rebound tonometer (Icare LAB system). At the conclusion of the sixth week of treatment, eye tissue and optic nerve tissue are harvested. Retinas are stained with gamma-synuclein, which preferentially stains RGCs. Cells are counted using a laser-scanning confocal microscope as previously described (Stothert, A. R., et al., 2017). Eye tissue can be lysed for analysis by Western blot. Alterations to the levels and assembly of Collagen IV, Collagen I, and Fibronectin can be assessed by denaturing and non-denaturing Western blot conditions, respectively.

Expected outcomes and alternative approaches: Precedent in the literature suggests that interventions to mature deoxycholate insoluble ECM is possible prior to ECM maturation (Filla, M. S., et al., Exp Eye Res, 165, 7-19 (2017)). Data described herein indicate that the inhibition of Grp94 prior to the formation of aberrant ECM, driven by dexamethasone, can prevent the changes which inevitably lead to elevated IOP. By examining these changes under both conditions of pre- and post-treatment (relative to the administration of dexamethasone), insight into the causality of these ECM changes can be gained. Similarly, by using LC-MS/MS, changes occurring in these cells and tissues can be more efficiently monitored and components of this pathology not yet considered can be identified. These studies provide mechanistic insight into the roles of PPIB and Hsp47; two ER resident chaperones that remain largely unexplored in glaucoma.

In vivo experiments on models of secondary glaucoma reveals if novel Grp94 inhibitors with improved efficacy and ocular properties are successful, while simultaneously validating the preliminary results. Analogue concentrations necessary for these experiments are adjusted if the PK and cellular efficacy studies performed as described previously, demonstrate significant improvements over 4Br—BnIm Novel 4br—BnIm analogues are shown to be successful at ameliorating the IOP phenotype observed in the preliminary experiments, and selective inhibition of Grp94 is successful if administered after the development of steroid-induced glaucoma. Additional animal experiments in which 4Br—BnIm or an analogue is applied after the generation of a dexamethasone-induced IOP phenotype are conducted.

Summary: Current therapies for glaucoma offer only symptom management, whereas the approach described herein targets disease pathogenesis which results in a new suite of Grp94-selective inhibitors. As described herein, selective inhibition of Grp94 can be used as an effective treatment for steroid-induced ocular hypertension and glaucoma.

### Example 4

### Grp94 is Fundamental Intermediary in the Development of Steroid-Induced Glaucoma

The endoplasmic reticulum (ER) resident Hps90 family member, Grp94 (glucose regulated protein 94kDa), regulates the stability and folding of proteins translated in the ER. However, these processes can become aberrant under stress conditions or in the presence of mutated aggregation-prone proteins. Grp94 has been identified as essential in the aggregation and subsequent toxicity of mutant forms of the myocilin protein; the pathogenic factor in certain forms of juvenile open-angle glaucoma. Steroid treatment regimens can cause secondary open-angle glaucoma and are known to induce myocilin expression. In this example, the role of Grp94 in the development of steroid-induced glaucoma (SIG) as a regulator of myocilin and protein that comprise the extracellular matrix (ECM) was examined. The results demonstrate a clear role for Grp94 in the pathogenesis of SIG and as a therapeutic target to prevent this disorder.

It has been demonstrated that glucose regulated protein 94 (Grp94), is responsible for the intracellular accumulation of mutant myocilin. Grp94 is a molecular chaperone responsible for the proper folding and localization of proteins translated in the rough endoplasmic reticulum (ER), destined for the extracellular space. When Grp94 is inhibited, through the administration of selective small-molecule inhibitors, mutant MYOC is cleared in both cell culture models as well as in vivo models of myocilin accumulation. By directly inhibiting Grp94, the pathology driving myocilin-induced glaucoma is ablated, unlike current glaucoma therapeutic strategies that only address humor production or outflow. Also demonstrated is that Grp94 regulates wild-type non-mutated myocilin under conditions of cellular stress. Studies into the mechanisms of SIG have suggested that cellular stress is a major factor in SIG pathogenesis. Together, these finding led to the hypothesis that steroid regimens can cause an aberrant stress response dependent on Gpr94.

Described in this example is the role of Grp94 in the development of steroid-induced glaucoma. Grp94 is necessary for dexamethasone to increase the intraocular pressure of dexamethasone treated mice. An examination of the underlying mechanisms suggest that Grp94 regulates components of the extracellular matrix; including Collagen I (COL1A1) and Fibronectin (FN1). Though some mechanistic questions remain, these studies demonstrate the dependence of steroid-induced glaucoma phenotypes on Grp94 and highlight the potential of Grp94 as a therapeutic target to prevent the development of steroid-induced glaucoma.

Materials and Methods

4Br—BnIm synthesis: 4Br—BnIm was synthesized as previously described (Crowley, V. M., et al. Journal of medicinal chemistry 59, 3471-3488, (2016)).

Animal Husbandry: Mice were housed and bred at the University of South Florida Byrd Alzheimer's Institute. All animal procedures performed in this study followed the ARVO Statement for the Use of Animals in Ophthalmic and Vision Research and were approved by the University of South Florida Institutional Animal Care and Use Committee. A cohort of 35 animals from separate litters were used for this study; 20 male mice (10 WT and 10 Myoc-KO) and 15 female mice (8 WT and 7 Myoc-KO). Total numbers of mice at the start of the experiment for each group: WT vehicle+vehicle (n=4), WT vehicle+4Br—BnIm (n=4), WT Dex+vehicle (n=5), WT Dex+4Br—BnIm (n=5), Myoc-KO vehicle+vehicle (n=4), Myoc-KO vehicle+4Br—BnIm (n=4), Myoc-KO Dex+vehicle (n=4), Myoc-KO Dex+4Br—BnIm (n=5). Technicians performing the drug delivery and tonometry measurements were blinded for animal genotype as well as each compound and the respective vehicle treatment. One Myoc-KO mice on the Dex+4Br—BnIm treatment was found dead two weeks prior to the final tonometry measurement and one additional Myoc-KO mice on the Dex+4Br—BnIm treatment had to be sacrificed the week of the final tonometry measurement due to wounds from fighting with a cagemate. No other treatment groups or geneotypes suffered losses. One WT animal on the Dex+4Br—BnIm study began the study with only one eye. Topical Ocular Delivery of 4Br—BnIm and Dexamethasone: Mice were treated 1×/day for 8 weeks with 4Br—BnIm and 3×/day for 7 weeks with 0.1% dexamethasone; appropriate controls were also applied on the same schedule. 4Br—BnIm treatment dose was based on previous studies. Once a day at 2 PM, mice were restrained and 1 drop (≈10 μL) of 4Br—BnIm or vehicle (DMSO in PBS) at 300 μM was applied topically to each eye. The drop was allowed to sit on the eye for 1 minute before the mouse was returned to its cage. One drop of 0.1% Dexamethasone eye drops (Dexamethasone Sodium Phosphate Ophthalmic Solution USP, 0.1% Dexamethasone Phosphate Equivalent; Bauch and Lomb) or PBS (vehicle) was applied to each eye. Dexamethasone treatments were conducted at 6:30 AM, 9 AM, and 2 PM, every day.

IOP Measurements: IOP levels in the mouse eye were obtained using the Icare TonoLab rebound tonometer and their guidelines were followed (Icare, Finland). Measurements were taken at LOAM to control for diurnal fluctuations in IOP levels which occur throughout the day. Mice were anesthetized with 3-4% isoflurane in oxygen once a week. After mice were sufficiently anesthetized, the mice were placed in an open restraint platform and IOP measurements were taken for each eye. The mice were anesthetized for no longer than two minutes during the acquisition of IOP. All mice were housed in the same housing room in the University of South Florida Byrd Alzheimer's Institute. All IOP measurements were conducted in the same procedure room, also in the University of South Florida Byrd Alzheimer's Institute.

Eye enucleation: Mice were euthanized with a 0.2% Somnasol (50 mg/kg) in saline solution. Eyes were gently removed from the skull preserving the morphology of the eye globe. Once removed from the skull, eyes were bisected into anterior and posterior sections and frozen for Western blot analysis.

HTM Treatments: Low passage number human trabecular meshwork (HTM) cells were cultured in DMEM supplemented with 10% fetal bovine serum and 1% penicillin/streptomycin. HTM cells were treated for 10 days (replaced every 2-3 days) with 100 nM dexamethasone. HTM cells were validated by expression of myocilin under these conditions. HTM cells treated with both dexamethasone and 4Br—BnIm received 4Br—BnIm in culture media for final 24 or 48 hours (where indicated) of the 10 day 100nM dexamethasone treatment.

Western and Dot Blots: Anterior eye segments and HTM cells were lysed in RIPA buffer. HTM lysates were separated by SDS-PAGE and transferred to a PVDF membrane. Lysates and culture media for dot blots were applied to a nitrocellulose membrane. Blots were blocked in 7% non-fat milk solution. Myocilin antibody was provided by W. D. Stamer. Collagen I and Fibronectin antibodies were purchased from Santa Cruz Biotechnology. Actin antibody was purchased from Sigma Aldrich (St. Louis, Mo.). Blots were developed using species specific secondary antibodies from Southern Biotech.

Quantification and Statistical Analysis: Two-tailed student's t-test and two-way ANOVA with Bonferroni's post-hoc tests were used as detailed in the figure legends. Values were considered significant if p<0.05. Graphs were generated using GraphPad Prism 5.0 analysis software or Microsoft Excel. N-value in this study is depicted as number of animals or number of eyes where indicated. Graphs are plotted as ±SEM or % CV as indicated.

Grp94, but not myocilin, necessary for steroid-induced IOP elevation: The results showing the regulation of mutant myocilin in genetic models of juvenile open-angle glaucoma suggested that Grp94 may be critical in the development of other forms of glaucoma; particularly, in steroid-induced glaucoma since myocilin is known to be induced by glucocorticoids. To assess this in vivo, a mouse model of steroid-induced glaucoma was utilized and a selective inhibitor of Grp94 (treatment strategy visualized in FIG. 1A). Wild-type (WT) mice treated three times-per-day, topically to the eye, with dexamethasone (dex, a corticosteroid) developed a statistically significant increase in IOP in two weeks; an increase of roughly 33% (FIG. 1B). Interestingly, the increased IOP was dependent on Grp94 activity. By ablating the activity of Grp94, prior to the steroid regimen, the steroid-induced IOP phenotype was prevented and IOP levels were statistically indistinguishable from control animals. Additionally, inhibition of Grp94 activity did not significantly change the IOP of non-dex treated mice; indicating that Grp94 is not a global regulator of aqueous humor production or outflow.

Results demonstrate that Grp94 regulates mutant myocilin and WT myocilin under stress conditions. Thus, it was hypothesized that Grp94 is promoting steroid-induced glaucoma through an aberrant regulation of myocilin. To address this, mice lacking myocilin (Myoc-/-, Myoc-KO) was treated with dexamethasone identically to the study in WT mice. Dex administration produced a statistically significant IOP increase three weeks after dex treatment initiation; this, despite the lack of myocilin (FIG. 1C). Ablating Grp94 activity in the Myoc-KO mice, prior to the initiation of dex treatment, prevented the IOP phenotype (similar to the WT mice). The measured IOP in the Grp94 inhibitor-treated mice was not statistically significant from non-dex treated mice; this, again demonstrating that Grp94 is not a global regulator of IOP.

Previous studies have indicated that, in certain dex-induced SIG models, steroid regimens can produce IOP phenotypes in Myoc-KO mice. However, in the SIG model described herein, WT mice developed an IOP phenotype two weeks after initiation of the dex treatment whereas the Myoc-KO mice presented a statistically significant IOP increase three weeks after treatment.

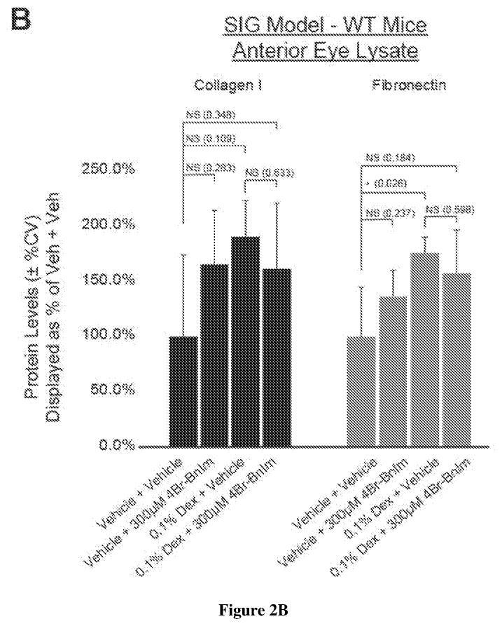

Grp94 regulates ECM components in SIG: Since the regulation of myocilin by Grp94 was not responsible for the elevated IOP or the rescue of the SIG IOP phenotype, Grp94-dependent changes to ECM components (as these proteins are implicated as necessary for the development of SIG) were assessed. Lysates of the anterior section of mice eyes, collected at the termination of the study, revealed a significant increase (N=8, p=0.026) in the levels of Fibronectin in the dex treated group (FIGS. 2A and B). A positive trend in the Collagen I levels of the dex treated group and a decrease in the mean values of both Collagen I and Fibronectin in the dex & 4Br—BnIm treated groups were observed. However, these changes were, overall, not statistically significant due to high variability in the samples.

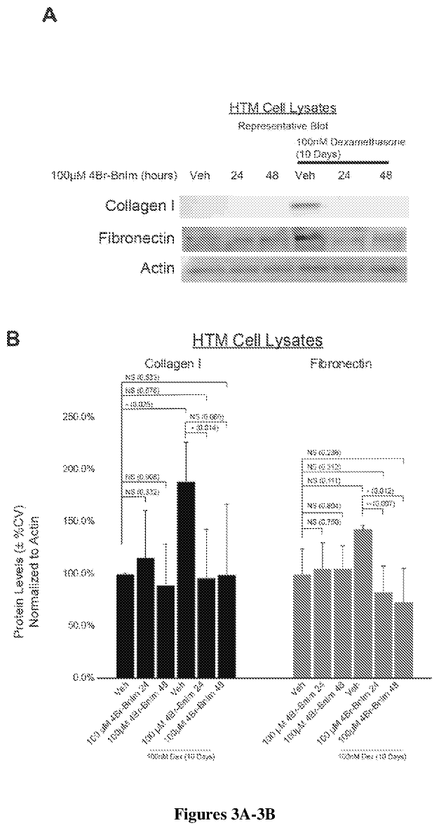

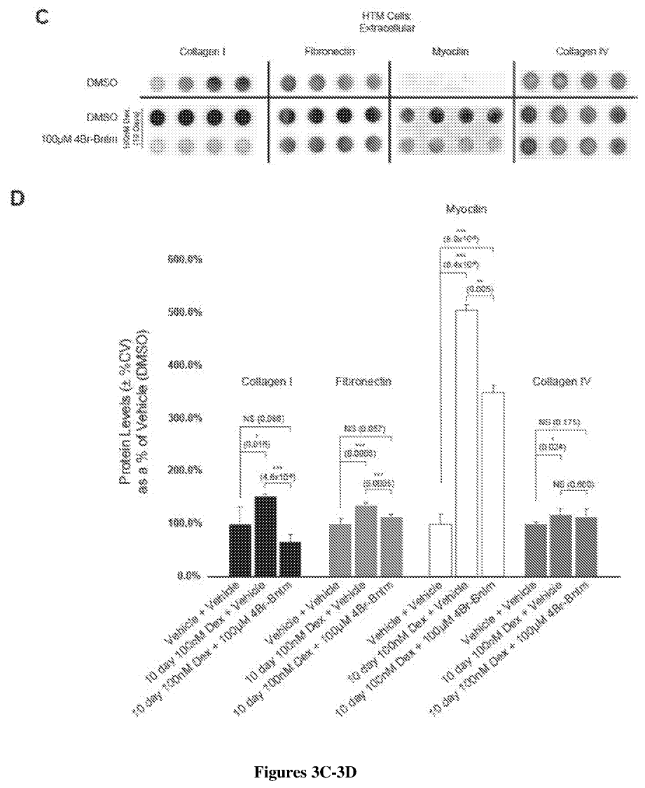

The high variability of the protein levels extracted from the mice eyes only suggested, but did not demonstrate, a clear cause and effect for the role of Grp94 in SIG. To address this, human trabecular meshwork cells (HTM cells) were used to assess changes to ECM proteins in a homologous system. The levels of both Collagen I and Fibronectin in HTM cell lysates were asayed. Additionally, the growth medium was collected from these treated HTM cells to assess the extracellular release of collagens and Fibronectin. Lysates from HTM cells treated for 10 days with 100 nM dexamethasone demonstrated statistically significant increases in internal Collagen I levels (p=0.025; repeated measures of duplicated experiments) and elevated levels of internal Fibronectin (not significant; p=0.111)(FIGS. 3A & B). These results were in-line with previous results. However, Grp94 inhibition in HTM cells treated with dex produced statistically significant decreases in the levels of both Collagen I and Fibronectin 24 hours after treatment (p=0.014 and p=0.007, respectively). Intracellular Collagen I and Fibronectin were unchanged in cells treated with 4Br—BnIm in the absence of dexamethasone.

Analysis of the extracellular levels of Collagen I and Fibronectin revealed statistically significant increases in extracellular Collagen I and Fibronectin (p=0.016 and p=0.0005, respectively); reflecting dexamethasone increasing both Collagen I and Fibronectin (FIGS. 3C and D). Inhibition of Grp94, in the presence of dexamethasone, significantly decreased the levels of both Collagen I and Fibronectin (p=4.6×10⁻⁶ and p=0.0005, respectively) to levels indistinguishable to vehicle treated cells (p=0.086 and p=0.057, respectively).

Additionally, myocilin was examined to both validate the HTM model and assess changes to other extracellular proteins. Dexamethasone increased extracellular myocilin by over 500% (p=6.4×10⁻⁶) and inhibition of Grp94 significantly reduced the amount of extracellular myocilin (p=0.005). Levels of Collagen IV, another ECM component, were moderately increased by dexamethasone; but remained unchanged by inhibition of Grp94. These data indicate that dexamethasone promotes an aberrant increase in the protein levels of both Collagen I and Fibronectin that is dependent on Grp94 activity.

Discussion

This work relates Grp94 and myocilin to a distinct form of secondary glaucoma. The results from in vivo tonometry experiments clearly demonstrate the necessity of Grp94 for the development of steroid-induced glaucoma.

It has been demonstrated that aberrant ECM components contribute to the development of SIG. The induction and subsequent reduction observed in this example suggests that, once induced through steroid regimens, both Collagen I and Fibronectin become hyper-dependent on, or addicted to, Grp94. It is believed there are factors necessary for the proper folding and release of Collagen I and Fibronectin that are limited in the ER. Induction in the levels of both Collagen I and Fibronectin, without a corresponding induction to these unidentified limiting factors, could yield an unstable or incompletely folded Fibronectin or Collagen I intermediate. This intermediate, once stabilized by Grp94, could be released in an incomplete state from the cell and generate the aberrant ECM observed in SIG.

This example demonstrates that ablating the activity of Grp94 prior to the administration of steroids was sufficient to prevent steroid-induced IOP elevations. It is possible that inhibiting Grp94 activity in the presence of existing SIG pathology will be able to restore the proteostasis of the afflicted TM cells. It is possible that Grp94 inhibitors are suitable treatments for existing SIG cases; however, this example demonstrates that it is suitable as a prevention mechanism. The data collected from HTM cells suggest treating existing SIG cases is possible since both Collagen I and Fibronectin were cleared in cells that had been treated with dexamethasone prior to the inhibition of Grp94.

These examples show in vivo success for Grp94 inhibitors in the treatment of glaucoma. Topical administration of Grp94 inhibitors are well tolerated with no noted toxicities. Similarly, no adverse events were observed throughout the in vivo studies. The lead molecule, 4Br—BnIm, requires minimal formulation to demonstrate efficacy in models of glaucoma. Medicinal chemistry efforts to improve the BnIm scaffold can be used to increase affinity for Grp94 and to improve uptake into the eye. Such modification would reduce the concentration, duration of treatment regimen, and total number of treatments necessary to treat mutant myocilin-dependent glaucoma and SIG. A detailed analysis of ocular pharmacokinetics and pharmacodynamics will also improve our treatment paradigm.

The disclosures of all publications cited above are expressly incorporated herein by reference, each in its entirety, to the same extent as if each were incorporated by reference individually.

It is also to be understood that the following claims are intended to cover all of the generic and specific features of the disclosure herein described, and all statements of the scope of the disclosure which, as a matter of language, might be said to fall there between. Now that the disclosure has been described.

1. A method for preventing, treating, or reducing steroid-induced ocular hypertension or glaucoma in a patient, comprising: administering a therapeutically effective amount of a Grp94-selective inhibitor to the patient in need thereof.

2. The method of claim 1, wherein the Grp94-selective inhibitor is a compound having a structure represented by Formula II or a pharmaceutically acceptable salt, solvate, derivative, or prodrug thereof: 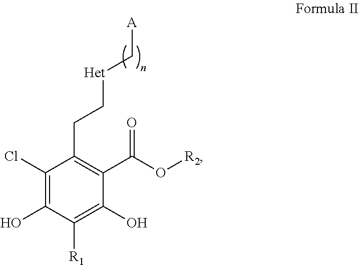
wherein
“Het” is a 5 membered heterocyclic moiety selected from a diazole moiety; or a triazole moiety;
A is a substituted or unsubstituted C₆-C₁₀ aryl or substituted or unsubstituted C₃-C₁₀ heteroaryl;
R₁ and R₂ are independently selected from hydrogen, halogen, C₁-C₆ alkyl, C₁-C₆ alkoxy, C₁-C₆ alkyl ether, C₁-C₆ alkyl halide, C₁-C₆ hydroxyalkyl, C₁-C₆ amino alkyl, C₃-C₆ cycloalkyl, or C₃-C₆ heterocycloalkyl; and
n is an integer from 1 to 5.

3. The method of claim 2, wherein A is a substituted or unsubstituted C₆ aryl or a substituted or unsubstituted C₃-C₆ heteroaryl.

4. The method of claim 2, wherein the Grp94-selective inhibitor is a compound having a structure represented by Formula IIA: 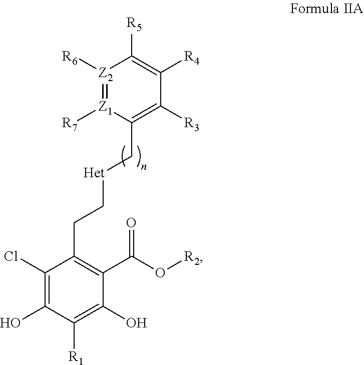
wherein
R₁ and R₄ are independently selected from hydrogen, halogen, or C₁-C₃ alkyl;
R₂ is C₁-C₆ alkyl;
R₃ is hydrogen, halogen, hydroxyl, amino, cyano, nitro, C₁-C₆ alkyl, C₁-C₆ alkoxy, C₁-C₆ hydroxyalkyl, or C₁-C₆ aminoalkyl;
Rs is halogen, C₁-C₆ alkyl, C₂-C₆ alkenyl, C₂-C₆ alkynyl, C₁-C₆ alkoxy, C₁-C₆ alkyl halide, hydroxyl, amino, cyano, or nitro;
R₆ and R₇ are independently present or absent and are independently selected from hydrogen, halogen, hydroxy, C₁-C₆ alkyl, C₁-C₆ alkoxy, C₁-C₆ alkyl halide, C₁-C₆ hydroxyalkyl, C₁-C₆ aminoalkyl;
Z₁ and Z₂ are independently selected from C or N.

5. The method of claim 1, wherein the Grp94-selective inhibitor is a compound having a structure represented by Formula IIA-1: 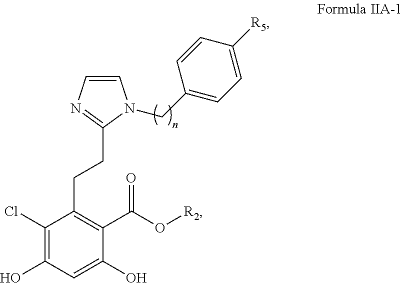
wherein
R₂ is C₁-C₆ alkyl;
R₅ is a halogen, preferably chloro or bromo; and
n is 1, 2, 3 or 4.

6. The method of claim 2, wherein n is 1.

7. (canceled)

8. The method of claim 4, wherein R₅ is chlor or bromo.

9. The method of claim 1, wherein the Grp94-selective inhibitor is methyl 2-(2-(1(4-bromobenzyl)-1H-imidazol-2-yl)ethyl)-3-chloro-4,6-dihydroxybenzoate (4-Br—BnIm) or a derivative thereof.

10. The method of claim 1, wherein the Grp94-selective inhibitor is administered prior to administering a steroid to the patient.

11. The method of claim 1, wherein the method prevents or reduce steroid-induced ocular hypertension or glaucoma in the patient.

12. (canceled)

13. (canceled)

14. The method of claim 1, wherein the patient has steroid-induced ocular hypertension.

15. The method of claim 1, wherein the patient has steroid-induced glaucoma.

16. (canceled)

17. The method of claim 1, wherein the Grp94-selective inhibitor is administered transdermally or topically.

18. The method of claim 1, wherein the Grp94-selective inhibitor is administered topically to the eye.

19. (canceled)

20. An ophthalmic solution comprising a therapeutically effective amount of a Grp94-selective inhibitor for the treatment of steroid-induced ocular hypertension or glaucoma and a pharmaceutically acceptable vehicle.

21. An ophthalmic solution comprising a Grp94-selective inhibitor which is a compound having a structure represented by Formula II or a pharmaceutically acceptable salt, solvate, derivative, or prodrug thereof: 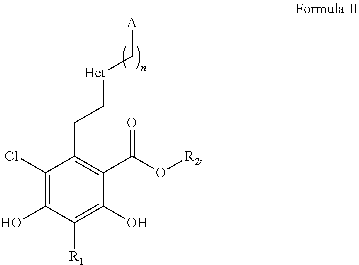
wherein
“Het” is a 5 membered heterocyclic moiety selected from a diazole moiety; or a triazole moiety;
A is a substituted or unsubstituted C₆-C₁₀ aryl or substituted or unsubstituted C₃-C₁₀ heteroaryl;
R₁ and R2 are independently selected from hydrogen, halogen, C₁-C₆ alkyl, C₁-C₆ alkoxy, C₁-C₆ alkyl ether, C₁-C₆ alkyl halide, C₁-C₆ hydroxyalkyl, C₁-C₆ amino alkyl, C₃-C₆ cycloalkyl, or C₃-C₆ heterocycloalkyl; and
n is an integer from 1 to 5.

22. The ophthalmic solution of claim 21, wherein A is a substituted or unsubstituted C₆ aryl or a substituted or unsubstituted C₃-C₆ heteroaryl, wherein the heteroatom in the heteroaryl is selected from N or O, and the substituent on the aryl or heteroaryl includes a halogen.

23. The ophthalmic solution of claim 21, wherein the Grp94-selective inhibitor is a compound having a structure represented by Formula IIA: 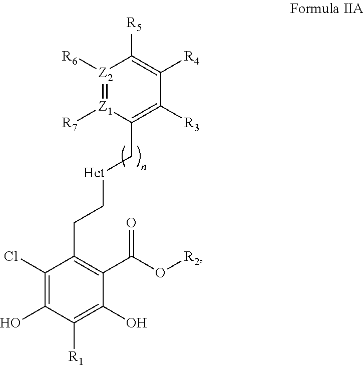
wherein
R₁ and R4 are independently selected from hydrogen, halogen, or C₁-C₃ alkyl;
R₂ is C₁-C₆ alkyl;
R₃ is hydrogen, halogen, hydroxyl, amino, cyano, nitro, C₁-C₆ alkyl, C₁-C₆ alkoxy, C₁-C₆ hydroxyalkyl, or C₁-C₆ aminoalkyl;
Rs is halogen, C₁-C₆ alkyl, C₂-C₆ alkenyl, C₂-C₆ alkynyl, C₁-C₆ alkoxy, C₁-C₆ alkyl halide, hydroxyl, amino, cyano, or nitro;
R6 and R₇ are independently present or absent and are independently selected from hydrogen, halogen, hydroxy, C₁-C₆ alkyl, C₁-C₆ alkoxy, C₁-C₆ alkyl halide, C₁-C₆ hydroxyalkyl, C₁-C₆ aminoalkyl;
Z₁ and Z2 are independently selected from C or N.

24. The ophthalmic solution of claim 21, wherein the Grp94-selective inhibitor is a compound having a structure represented by Formula IA-1: 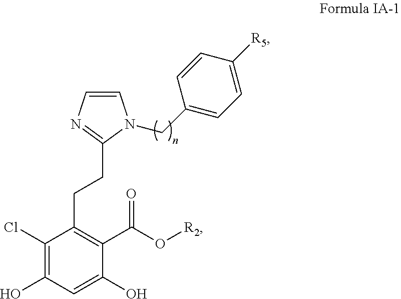
wherein
R₂ is C₁-C₆ alkyl;
R₅ is a halogen, preferably chloro or bromo; and
n is 1, 2, 3 or 4.

25. (canceled)

26. (canceled)

27. (canceled)

28. (canceled)

29. The ophthalmic solution of claim 20, comprising 0.1% to 2% by weight of the active ingredient, based on the weight of the ophthalmic solution.
